(19) United States
(12) Patent Application Publication
(10) Pub. No.: US 2024/0382519 A1
(43) Pub. Date: Nov. 21, 2024

(54) MONOVALENT COPPER/PORPHYRIDIUM SP. POLYSACCHARIDE COMPLEXES, RELATED PRODUCTS AND METHODS OF PREPARATION THEREOF

(71) Applicant: B.G. NEGEV TECHNOLOGIES AND APPLICATIONS LTD., AT BEN-GURION UNIVERSITY, Beer Sheva (IL)

(72) Inventors: Shoshana ARAD, Omer (IL); Ariel KUSHMARO, Beer Sheva (IL); Nofar YEHUDA, Dimona (IL)

(21) Appl. No.: 18/273,467

(22) PCT Filed: Jan. 4, 2022
(86) PCT No.: PCT/IL2022/050014

§ 371 Date: Jul. 20, 2023

Related U.S. Application Data
(60) Provisional application No. 63/141,515, filed on Jan. 26, 2021.

(51) Int. Cl. A61K 33/34; A61K 36/04; A61P 31/02

(52) U.S. Cl. CPC A61K 33/34; A61K 36/04; A61P 31/02

(57) ABSTRACT

A composition including a monovalent copper compound and Porphyridium sp. Polysaccharide (PS). Methods of making the composition are also disclosed.

### FIELD OF THE INVENTION

The invention is in the field of antifungal and/or antibacterial compounds

### BACKGROUND OF THE INVENTION

The eight genera of red microalgae are morphologically the simplest of all red algae. red microalgae are found in fresh water as well as brackish water or seawater. Red microalgae reproduce asexually and most species appear brown as a result of their chlorophyll and phycoerythrin contents.

Cells of the red microalga Porphyridium sp. are encapsulated within a sulfated polysaccharide. This sulfated polysaccharide is composed of ten different sugars including xylose, glucose and galactose in significant amounts. The sulfated polysaccharide has a molecular mass of 5-7×10⁶ Da and is negatively charged. The sulfate content in the polysaccharide varies between 6 and 7% w/v. Aqueous solutions of the polysaccharide are stable over a wide range of temperatures (30-160° C.), pH values (2-9), and salinities, and solution viscosity is unaffected by changes in the environment.

The sulfated polysaccharide provides a buffer layer around the cells, protecting them against severe environmental conditions and may also contribute to cellular ionic regulation by selective cation binding. The Porphyridium sp. sulfated polysaccharide exhibits a variety of bioactivities, including anti-viral, anti-inflammatory, anti-oxidant, anti-biofilm, and bio-lubricant activities.

### SUMMARY OF THE INVENTION

A broad aspect of the invention relates complexes of metal with Porphyridium sp. sulfated polysaccharide

One aspect of some embodiments of the invention relates to a complex containing monovalent Cu ions complexed to Porphyridium sp. sulfated polysaccharide (PS) (e.g., Cu₂O-PS or CuCl-PS). According to various exemplary embodiments of the invention the complex is employed in bandages, wound dressings, topical medications (e.g., creams and/or ointments), as a coating in biofilm driven packaging, and as an anti-biofilm coating on medical devices (e.g., catheters). In some exemplary embodiments of the invention, the broad biocidal activity of the monovalent Cu-PS complex mitigates a need for concurrent application of separate antibacterial and antifungal agents.

It will be appreciated that the aspect described above relates to solution of technical problems associated with development of antibiotic resistant strains of bacteria and/or fungi.

Alternatively or additionally, it will be appreciated that the aspect described above relates to solution of technical problems related to a need to administer separate antifungal and antibacterial compounds.

In some exemplary embodiments of the invention there is provided a composition including: (a) a monovalent copper compound; and (b) Porphyridium sp. Polysaccharide (PS). In some embodiments, the monovalent copper compound includes Cu₂O (cuprous oxide). Alternatively or additionally, in some embodiments the monovalent copper compound includes CuCl (cuprous chloride). Alternatively or additionally, in some embodiments the composition includes the monovalent copper at a concentration ≥20 PPM. Alternatively or additionally, in some embodiments the composition includes the monovalent copper at a concentration ≤750 PPM. Alternatively or additionally, in some embodiments the composition includes the PS at a concentration ≥0.05% (w/v). Alternatively or additionally, in some embodiments the composition includes the PS at a concentration ≤1.0% (w/v). Alternatively or additionally, in some embodiments the composition is provided in a form selected from the group consisting of a spray, a cream and an ointment. Alternatively or additionally, in some embodiments the composition is provided as part of wound dressing including the composition as described above on an area that contacts a wound when the dressing is in use. Alternatively or additionally, in some embodiments the composition is applied to a medical device.

In some exemplary embodiments of the invention there is provided a method including: (a) adding a monovalent copper compound to a solution of Porphyridium sp. Polysaccharide (PS); and (b) dissolving the monovalent copper compound in the solution to produce an antifungal solution including dissolved complex of monovalent copper/PS. In some embodiments, the method includes sterilizing the antibiotic solution. Alternatively or additionally, in some embodiments the monovalent copper compound includes Cu2O (cuprous oxide). Alternatively or additionally, in some embodiments the monovalent copper compound includes CuCl (cuprous chloride). Alternatively or additionally, in some embodiments the antibiotic solution includes monovalent copper at a concentration ≥20 PPM. Alternatively or additionally, in some embodiments the antibiotic solution includes monovalent copper at a concentration ≤750 PPM. Alternatively or additionally, in some embodiments the antibiotic solution includes the PS at a concentration ≥0.05% (w/v). Alternatively or additionally, in some embodiments the antibiotic solution includes the PS at a concentration ≤1.0% (w/v). Alternatively or additionally, in some embodiments the method includes incorporating the antibiotic solution in a topical administration form selected from the group consisting of a spray, a cream and an ointment. Alternatively or additionally, in some embodiments the method includes incorporating the antibiotic solution into/onto a wound dressing at an area that contacts a wound. Alternatively or additionally, in some embodiments the method includes applying the antibiotic solution to a surface.

Unless otherwise defined, all technical and scientific terms used herein have the same meaning as commonly understood by one of ordinary skill in the art to which this invention belongs. Although suitable methods and materials are described below, methods and materials similar or equivalent to those described herein can be used in the practice of the present invention. In case of conflict, the patent specification, including definitions, will control. All materials, methods, and examples are illustrative only and are not intended to be limiting.

As used herein, the terms “comprising” and “including” or grammatical variants thereof are to be taken as specifying inclusion of the stated features, integers, actions or components without precluding the addition of one or more additional features, integers, actions, components or groups thereof. This term is broader than, and includes the terms “consisting of” and “consisting essentially of” as defined by the Manual of Patent Examination Procedure of the United States Patent and Trademark Office. Thus, any recitation that an embodiment “includes” or “comprises” a feature is a specific statement that sub embodiments “consist essentially of” and/or “consist of” the recited feature.

The phrase “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

The phrase “adapted to” as used in this specification and the accompanying claims imposes additional structural limitations on a previously recited component.

The term “method” refers to manners, means, techniques and procedures for accomplishing a given task including, but not limited to, those manners, means, techniques and procedures either known to, or readily developed from known manners, means, techniques and procedures by practitioners of architecture and/or computer science.

Percentages (%) of chemicals are w/v (weight per volume) unless otherwise indicated.

### BRIEF DESCRIPTION OF THE DRAWINGS

In order to understand the invention and to see how it may be carried out in practice, embodiments will now be described, by way of non-limiting example only, with reference to the accompanying figures. In the figures, identical and similar structures, elements or parts thereof that appear in more than one figure are generally labeled with the same or similar references in the figures in which they appear. Dimensions of components and features shown in the figures are chosen primarily for convenience and clarity of presentation and are not necessarily to scale. The attached figures are:

FIG. 1 is a plot of FT-IR the transmission spectra (absorbance units (a.u.) as a function of wavelength (cm⁻¹)) of polysaccharide (PS) and the Cu₂O-PS complex (0.7% polysaccharide (w/v) and 500 ppm copper) in the region of 650-4000 cm⁻¹;

FIG. 2a is an SEM micrograph (600×) of 0.7% PS (w/v) alone;

FIG. 2b is an SEM micrograph (600×) of Cu₂O-PS complex in 0.7% PS (w/v) at a copper concentration of 500 PPM;

FIG. 2c is an EDS spectrum (intensity counts as a function of energy (eV)) of the polysaccharide sample of FIG. 2a (area 1);

FIG. 2d-1 is an EDS spectrum (intensity counts as a function of energy (eV)) of the Cu₂O-PS complex sample of FIG. 2b in a smooth area (area 1);

FIG. 2d-2 is an EDS spectrum (intensity counts as a function of energy (eV)) of the Cu₂O-PS complex sample of FIG. 2b in a spiked area (area 2);

FIG. 3a1 an AFM surface topography and 3D image of 0.7% (w/v) PS and showing smooth surface of the polysaccharide;

FIG. 3a2 is a 2D SEM image of 0.7% (w/v) PS;

FIG. 3a3 is a 2D AFM image of 0.7% (w/v) PS;

FIG. 3b1 is an AFM surface topography and 3D image Cu₂O-PS complex (0.7% (w/v) PS and 500 ppm copper) showing needle-like structures of the complex;

FIG. 3b2 is a2D SEM image Cu₂O-PS complex (0.7% (w/v) PS and 500 ppm copper);

FIG. 3b3 is a2D AFM image Cu₂O-PS complex (0.7% (w/v) PS and 500 ppm copper);

FIG. 4 is a bar graph of % viability C. albicans, A. baumannii, Pseudomonas aeruginosa, E. coli, S. aureus and B. subtilis cultures exposed to no treatment (black bar), 0.07% (w/v) PS (green bar), Cu₂O solution (30 PPM copper) (blue bar) and Cu₂O/PS complex solution [0.07% (w/v) PS and 30 PPM copper] with **** indicating a significant difference (p<0.05) by ANOVA test;

FIG. 5 is series of HR-SEM images of biofilm assays against C. albicans and Pseudomonas aeruginosa PA14 using clean glass surface (leftmost image in each row), PS only (0.7% w/v); Cu₂O (500 ppm) only and Cu₂O-PS complex (0.7% (w/v) PS and 500 ppm copper);

FIG. 6 is a cyclic voltammogram {I[μA] as a function of (E/V vs SCE [V])} of Cu₂O-PS complex (0.7% (w/v) PS and 500 ppm copper) in acetonitrile: GCE working, Ag/AgCl reference, and Pt auxiliary electrodes and lithium perchlorate as a supporting electrolyte; [lithium perchlorate]=2 M;

FIG. 7a is a mechanical spectra of PS (0.7% w/v) indicating G′ (storage modulus) and G″ (loss modulus) as a function of angular frequency (w) at 25° C.; FIG. 7b is a mechanical spectra of Cu₂O-PS complex (0.7% w/v PS and 500 ppm copper) indicating G′ (storage modulus) and G″ (loss modulus) as a function of angular frequency (ω) at 25° C.;

FIG. 8 is a plot of absorption coefficient [cm⁻¹] as a function of wavelength [nm] for PS (0.7% w/v; green line), Cu₂O-PS complex (0.7% w/v PS; 500 PPM copper; red line), and Cu₂O alone (500 ppm measured and calculated, solid and dashed blue lines, respectively); dashed blue line shows the Cu₂O curve calculated from the polysaccharide and Cu₂O-polysaccharide curves using Eq. 2;

FIG. 9 is a plot of Photoluminescence [a.u.] as a function of wavelength [nm] for PS 0.7% (w/v) (green), Cu₂O-PS complex (0.7% (w/v) polysaccharide and 500 ppm copper) (red) and Cu₂O 500 ppm (blue) showing that addition of Cu₂O to PS increased photoluminescence at wavelengths >800 nm;

FIG. 10 is a plot of OD (600 nm) as a function of time (hrs) for Candida albicans cultures exposed to Bifonazole (1 μg/mL; blue line), Cu₂O (30 ppm Cu; purple line), PS (0.07% w/v; green line), Cu₂O-PS complex (0.07% w/v PS with 30 ppm Cu; red line) and negative control culture (black line)

FIG. 11 is a plot of copper concentration in DI (ppm) as a function of time (hrs.) for Monovalent and Divalent Cu-PS complexes to DI medium: PS only (green line), Cu₂O-PS (red line with squares), CuCl-PS (red line with triangles), CuO-PS (blue line with triangles), CuCl₂-PS (blue line with diamonds), in this Fig. all Cu-PS complexes contain 0.7% (w/v) polysaccharide and 500 ppm copper;

FIG. 12 is a series of AFM surface topography and 3D images of the complexes Cu₂O-PS, CuCl-PS, CuO-PS, and CuCl₂-PS with relevant characteristics summarized numerically below;

FIG. 13 is a series of photomicrographs illustrating the influence of PS only (0.7% w/v), Cu₂O-PS (0.7% w/v PS and 500 ppm copper), CuCl-PS (0.7% w/v PS and 500 ppm copper), CuO-PS (0.7% w/v PS and 500 ppm copper), and CuCl₂-PS (0.7% w/v PS and 500 ppm copper) on growth of P. aeruginosa and C. albicans grown on glass plates with and without an additional layer of gold;

FIG. 14 is plot of luminescence [RLU] as a function of time in minutes for E. coli TV1061 induced to bioluminescence PS only (0.07% w/v; green line), Cu₂O-PS (0.07% w/v PS and 30 ppm copper; red line), CuCl-PS (0.07% w/v PS and 30 ppm copper; brown line), CuO-PS (0.07% w/v PS and 30 ppm copper; light blue line), and CuCl₂-PS (0.07% w/v PS and 30 ppm copper; dark blue line) and 20% ethanol (black line).

FIG. 15a is an SEM micrograph (600×) of a Cu₂O-PS complex [0.7% (w/v) polysaccharide and 500 ppm copper];

FIG. 15b is an EDS spectrum (Intensity (counts) as a function of Energy (eV)) of the Cu₂O-PS complex of FIG. 15a;

FIG. 15c is an SEM micrograph (600×) a CuCl-PS complex; [0.7% (w/v) polysaccharide and 500 ppm copper];

FIG. 15d is an EDS spectrum (Intensity (counts) as a function of Energy (eV)) of the CuCl-PS complex of FIG. 15c;

FIG. 15e is an SEM micrograph (600×) of a CuO-SP complex [0.7% (w/v) polysaccharide and 500 ppm copper];

FIG. 15f is an EDS spectrum (Intensity (counts) as a function of Energy (eV)) of the CuO-SP complex of FIG. 15e;

FIG. 15g is an SEM micrograph (600×) of a CuCl₂-PS complex [0.7% (w/v) polysaccharide and 500 ppm copper];

FIG. 15h is an EDS spectra (Intensity (counts) as a function of Energy (eV)) of the CuCl₂-PS complex of FIG. 15g;

FIG. 15i is an SEM micrograph (600×) of 0.7% (w/v) polysaccharide (PS);

FIG. 15j is an EDS spectra (Intensity (counts) as a function of Energy (eV)) of the PS of FIG. 15i; and

FIG. 16 is an FT-IR transmission spectra of PS (green line) and the Divalent Cu-complexes (CuO-PS and CuCl₂-PS; blue lines) and Monovalent Cu-complexes (Cu₂O-PS and CuCl-PS red lines) in the region of 650-4000 cm−1; all Cu-PS complexes contained 0.7% polysaccharide (w/v) and 500 ppm copper.

### DETAILED DESCRIPTION OF EMBODIMENTS

Embodiments of the invention relate to monovalent copper/polysaccharide compositions, methods of making the compositions and ways to use the compositions.

Specifically, some embodiments of the invention can be used to prevent or retard growth of fungi and/or bacteria or even to kill fungi and/or bacteria.

The principles and operation of compositions and methods according to exemplary embodiments of the invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

### Exemplary Composition

In some exemplary embodiments of the invention there is provided a composition including a monovalent copper compound and Porphyridium sp. Polysaccharide (PS). In some embodiments, the monovalent copper compound includes Cu₂O (cuprous oxide) and/or CuCl (cuprous chloride). Alternatively or additionally, in some embodiments the monovalent copper is present at a concentration ≥20 PPM. These values were calculated at first, the complexes were prepared and then the concentrations were validated using coupled plasma optical emission spectrometry (SPECTRO ARCOS ICP-OES analyzer). Alternatively or additionally, in some embodiments the monovalent copper is present at a concentration ≤750 PPM.

According to various exemplary embodiments of the invention the monovalent copper is present at a concentration ≥20 PPM, ≥30 PPM, ≥50 PPM, ≥50 PPM, ≥60 PPM, ≥70 PPM, ≥80 PPM, ≥90 PPM, ≥100 PPM, ≥150 PPM,≥200 PPM, ≥250 PPM, ≥300 PPM, ≥350 PPM, ≥400 PPM, ≥450 PPM, ≥500 PPM, ≥550 PPM,≥600 PPM, ≥650 PPM, ≥700 PPM, ≥750 PPM or intermediate or greater concentrations. Alternatively or additionally, according to various exemplary embodiments of the invention the monovalent copper is present at a concentration ≤750 PPM, ≤700 PPM, ≤650 PPM, ≤600 PPM, ≤550 PPM, ≤500 PPM, ≤450 PPM, ≤400 PPM, ≤350 PPM, ≤300 PPM, ≤250 PPM, ≤200 PPM, ≤150 PPM, ≤100 PPM, ≤50 PPM, ≤40 PPM, ≤30 PPM, ≤20 PPM or intermediate or lower concentrations.

Alternatively or additionally, the PS is present at a concentration ≥0.05% (w/v), ≥0.1% (w/v), ≥0.15% (w/v), ≥0.20% (w/v), ≥0.25% (w/v), ≥0.30% (w/v), ≥0.35% (w/v), ≥0.40% (w/v), ≥0.45% (w/v), ≥0.50% (w/v), ≥0.55% (w/v), ≥0.60% (w/v), ≥0.65% (w/v), ≥0.70% (w/v), ≥0.75% (w/v), ≥0.80% (w/v), ≥0.85% (w/v), ≥0.90% (w/v), ≥0.85% (w/v), ≥0.90% (w/v), ≥0.95% (w/v), ≥1.00% (w/v) or intermediate or greater concentrations. Alternatively or additionally, in some embodiments the PS is present at a concentration ≤1.0% (w/v), ≤0.95% (w/v), ≤0.90% (w/v), ≤0.85% (w/v), ≤0.80% (w/v), ≤0.75% (w/v), ≤0.70% (w/v), ≤0.65% (w/v), ≤0.60% (w/v), ≤0.55% (w/v), ≤0.50% (w/v), ≤0.45% (w/v), ≤0.40% (w/v), ≤0.35% (w/v), ≤0.30% (w/v), ≤0.25% (w/v), ≤0.20% (w/v), ≤0.15% (w/v), ≤0.10% (w/v), ≤0.05% (w/v) or intermediate or lower concentrations.

According to various exemplary embodiments of the invention the composition is provided in a form selected from the group consisting of a spray, a cream and an ointment. Some exemplary embodiments of the invention relate to a wound dressing comprising a composition as described above on an area that contacts a wound. For example, in some embodiments, the wound dressing is provided as an adhesive patch in a sterile wrapper with the monovalent Cu/PS-complex composition applied to a gauze pad that contacts the wound when the dressing is in use. In other exemplary embodiments of the invention, the monovalent Cu/PS-complex composition is applied to a medical device (e.g. a catheter).

### Exemplary Method

In some exemplary embodiments of the invention there is provided a method including adding a monovalent copper compound to a solution of Porphyridium sp. Polysaccharide (PS) and dissolving the monovalent copper compound in the solution to produce an antibiotic solution comprising dissolved complex of monovalent copper/PS. In some exemplary embodiments of the invention, the mixing occurs at room temperature. Alternatively or additionally, according to various exemplary embodiments of the invention the mixing lasts 0.25, 0.5, 1, 2, 4, 6, 8, 10, 12, 14, 16, 18, 20, 22, 24, 26, 28 or 30 hours or intermediate or longer times.

In some exemplary embodiments of the invention, the method includes sterilizing the antibiotic solution. In some embodiments, sterilization is by heat and/or pressure, for example in an autoclave.

In some exemplary embodiments of the invention, the monovalent copper compound includes Cu₂O (cuprous oxide) and/or CuCl (cuprous chloride). According to various exemplary embodiments of the invention the concentrations of monovalent copper are as described hereinabove. In some exemplary embodiments of the invention, the antibiotic solution comprises monovalent copper at a concentration ≥20 PPM. Alternatively or additionally, in some embodiments the antibiotic solution comprises monovalent copper at a concentration ≤750 PPM.

Alternatively or additionally, in some embodiments the antibiotic solution includes PS at a concentration ≥0.05% (w/v). Alternatively or additionally, in some embodiments the antibiotic solution includes PS at a concentration ≤1.0% (w/v). According to various exemplary embodiments of the invention the PS concentrations are as described hereinabove.

In some exemplary embodiments of the invention, the method includes incorporating the antibiotic solution in a topical administration form selected from the group consisting of a spray, a cream and an ointment. Alternatively or additionally, in some embodiments the method includes incorporating the antibiotic solution into/onto a wound dressing at an area that contacts a wound. In some exemplary embodiments of the invention, the wound dressing is an adhesive patch as described above. In some exemplary embodiments of the invention, the method includes applying the antibiotic solution to a surface. In some exemplary embodiments of the invention, the surface is the surface of a medical device (e.g. catheter). Application of the complex prevents growth of bacteria and/or fungi on the treated surface.

### Exemplary Advantages

Physicochemical and bioactivity characterization of Cu₂O-polysaccharide complex demonstrates that Cu ions were covalently bound to the polysaccharide in Cu₂O-polysaccharide complex as indicated by FT-IR.

The Cu₂O-polysaccharide complex exhibited higher viscosity and conductivity than the native polysaccharide, but zeta-potential and rheological properties were similar. Likewise, the complex displayed a porous fibrous structure and weak-gel-like behavior, similar to the native polysaccharide.

The Cu₂O-polysaccharide complex was effective against various microorganisms as compared to the polysaccharide or Cu alone. It almost completely inhibited C. albicans (91% inhibition) and presented significant inhibitory activity of biofilm formation against bacteria (Pseudomonas aeruginosa) and fungi (C. albicans), as seen from SEM images. The Cu₂O-polysaccharide complex was characterized by needle-like topographical protrusions (on AFM). The structures may be related to the complex's antimicrobial and antibiofilm activities. Although the EDS-SEM studies indicated the existence of Cu on the surface of the complex, the main anti-microbial effect appears to be from the spikes of 1,000 nm in height and 10-20 nm in width, at a density of about 5,000 spikes/μm².

The sulfated polysaccharide of Porphyridium sp. was used as a platform for the incorporation of Cu₂O.

Cu₂O and other metal-polysaccharide combinations with potential antibacterial and fungicidal activities show great promise in topical antimicrobial applications.

It is expected that during the life of this patent many Porphyridium algae strains will be developed and the scope of the invention is intended to include all such new strains a priori.

As used herein the term “about” refers to ±10%.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

Specifically, a variety of numerical indicators have been utilized. It should be understood that these numerical indicators could vary even further based upon a variety of engineering principles, materials, intended use and designs incorporated into the various embodiments of the invention. Additionally, components and/or actions ascribed to exemplary embodiments of the invention and depicted as a single unit may be divided into subunits. Conversely, components and/or actions ascribed to exemplary embodiments of the invention and depicted as sub-units/individual actions may be combined into a single unit/action with the described/depicted function.

Alternatively, or additionally, features used to describe a method can be used to characterize an apparatus and features used to describe an apparatus can be used to characterize a method.

It should be further understood that the individual features described hereinabove can be combined in all possible combinations and sub-combinations to produce additional embodiments of the invention. The examples given above are exemplary in nature and are not intended to limit the scope of the invention which is defined solely by the following claims.

Each recitation of an embodiment of the invention that includes a specific feature, part, component, module or process is an explicit statement that additional embodiments of the invention not including the recited feature, part, component, module or process exist.

Alternatively or additionally, various exemplary embodiments of the invention exclude any specific feature, part, component, module, process or element which is not specifically disclosed herein.

Specifically, the invention has been described in the context of wound dressings, creams and ointments but might also be used to retard fungal and/or bacterial growth on any surface.

All publications, references, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

The terms “include”, and “have” and their conjugates as used herein mean “including but not necessarily limited to”.

Additional objects, advantages, and novel features of various embodiments of the invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non-limiting fashion.

The following materials and methods are used in performance of experiments described in examples hereinbelow:

Algal Growth and Polysaccharide Production: Porphyridium sp. (UTEX 637) obtained from the culture collection of the University of Texas at Austin was grown in artificial seawater. Culture of the algal cells and isolation of the extracellular polysaccharide were performed as previously described (Cohen and Arad (1989) Biomass 18(1):59-67)

Briefly, the cells were grown in polyethylene sleeves in the appropriate medium. The cultures were illuminated continuously with fluorescent cool-white lamps at an irradiance of 150 μE m⁻²s⁻¹ and aerated with sterile air containing 3% CO₂. Cells were harvested at the stationary phase of growth by continuous centrifugation (CEPA, Carl Padberg Zentrifugenbau GmbH, Lahr, Germany). The supernatant containing the dissolved polysaccharide was collected and filtered using crossflow filtration to remove salts and other metabolites (MaxCell® hollow fiber microfiltration cartridge, pore size 0.45 μm, membrane area 2.5 m²) and concentrated to 0.7% (w/v) polysaccharide. The resulting polysaccharide was sterilized in an autoclave and stored at 4° C. Sugar content was determined using the phenol-sulfuric acid reaction (Dubois, M.; Gilles, K. A.; Hamilton, J. K.; Rebers, P. A.; Smith, F. Colorimetric Method for Determination of Sugars and Related Substances. 1956)

Complex Preparation: The Cu₂O-polysaccharide complex was prepared by directly adding Cu₂O (Fisher Scientific, Loughborough, UK) to 20 ml of a 0.7% (w/v) polysaccharide solution to give a final copper concentration of 500 ppm. The Cu₂O-polysaccharide complex was stirred gently with a magnetic stirrer for 24 hr at room temperature and then sterilized by autoclaving.

Copper Concentration: The copper concentration in the Cu₂O-polysaccharide complex was evaluated by inductively coupled plasma optical emission spectrometry (SPECTRO ARCOS ICP-OES analyzer).

Viscosity: The viscosity of the polysaccharide solutions was determined with a Brookfield digital viscometer, 30 rpm at room temperature with a 31 cylindrical spindle (Brookfield AMETEK SC4-31).

Conductivity: The conductivity of the polysaccharide solutions was determined with a pH/mV/Cond./TDS/Temp. meter 86505 at room temperature.

Cyclic Voltammetry: A Metrohm 757 VA Computerize instrument was employed to obtain cyclic voltammograms of the Cu₂O-polysaccharide complex in an acetonitrile solution at room temperature (25° C.) under a nitrogen atmosphere, with lithium perchlorate as the supporting electrolyte. A glass carbon working electrode, a platinum auxiliary electrode and an Ag/AgCl reference electrode were also used.

Rheology (G′, G″); Dynamic viscoelastic characterization of Cu₂O-polysaccharide complex (0.7% w/v polysaccharide with 500 ppm Cu) was determined by the frequency dependence of the storage and loss moduli, G′ and G″. Measurements were carried out using a Rheometer AR 2000 (TA Instruments), equipped with an extended temperature cell for temperature control and a stainless-steel parallel plate (d=40 mm). The samples were held at room temperature for at least 20 min. After the temperature reached 25° C., rheological tests were performed.

Fourier Transform Infrared (FT-IR) Spectroscopy: Spectra were obtained on an iN-10 FTIR ThermoFisher Microscope spectrometer equipped with a narrow-band liquid-nitrogen-cooled MCTA detector. Samples of the Cu₂O-polysaccharide complex and the polysaccharide were lyophilized at −55° C. for 24 h in 96-well plates. The spectra were recorded in three areas per sample in the range of 4000 to 650 cm⁻¹, at 2 cm⁻¹ resolution and 64 scans; area of detection was 25×25 mm. The FTIR data were collected using OMNIC Picta software. Automatic baseline correction was used.

Scanning Electron Microscopy/Energy Dispersive X-Ray Spectroscopy (SEM-EDS) Analysis: The morphological properties of the Cu₂O-polysaccharide complex and the native polysaccharide were analyzed by SEM (FEI ESEM Quanta 200) at an accelerating voltage of 20 kV. In preparation for SEM scanning, 200-μL samples were lyophilized at −55° C. for 24 h in 96-well plates. Then, the dried samples were attached to specimen holders with double-sided carbon tape and coated with a 20 nm layer of gold using the EMITECH K575x sputtering device (Emitech Ltd, UK).

Atomic Force Microscopy (AFM): Glass coverslips were immersed in 2.5 M HCl for 10 min and then rinsed, first with ethanol (99.9%) and then with distilled water. Then, 10 μL of the Cu₂O-polysaccharide complex or the native polysaccharide were applied to the glass surface and dehydrated by autoclaving at 121° C. for 40 min. Topographical images of the pre-dried Cu₂O-polysaccharide complex and native polysaccharide were acquired on a Dimension-3100 microscope (Bruker). Samples were imaged at a scan rate of 0.5-1 Hz with a 512×512 pixel resolution in tapping mode. Several scans were carried out over a given surface area. Potential measurements via scanning Kelvin probe microscopy (SKPM) used an MFP-3D-Bio inverted optical microscope system with an ARC2 controller (Asylum Research, Oxford Instruments). The images were recorded by SKPM in NAP mode, with a two-pass method. On the first path, the cantilever was oscillated mechanically, and the surface topography was recorded, while in the second path, during which AC and DC voltage were applied, the surface potential was recorded. It is important to note that the needle-like structures shown by the Cu₂O-polysaccharide complex in the AFM images were observed only under dry AFM conditions. AFM images were analyzed, and the needle like structures (spikes) were counted by using Gwyddion and ImageJ software (Elbourne et al. (2019) Nanoscale Adv. 1 (1):203-212).

Microbial Cultures and Growth Conditions: The antimicrobial and antibiofilm activities of the Cu₂O-polysaccharide complex was tested on a variety of model microorganisms cultured as follows: Acinetobacter baumannii, Escherichia coli and Pseudomonas aeruginosa PA14 were cultivated for 24 h in LB Broth (Miller) (Sigma-Aldrich). Staphylococcus aureus was grown in BD™ Tryptic Soy Broth (TSB; soybean-casein digest medium). Bacillus subtilis was cultivated in LB medium (Difco Luria-Bertani medium, Lennox). C. albicans (ATCC 10231, supplied by the Clinical Microbiology Laboratory of Dr. Yossi Paitan, Meir Medical Center, Kfar Saba) was cultivated in Potato Dextrose Broth (PDB; HiMedia) for 24 hr. with shaking (120 rpm) at a constant temperature of 37° C. For the antibiofilm study, Pseudomonas aeruginosa PA14 was inoculated into AB trace Minimal Medium, supplemented with 30 mm glucose at a constant temperature of 37° C.

Antimicrobial Activity: The antimicrobial activities of the Cu₂O-polysaccharide complex and of the native polysaccharide (in the form of a soft gel) against A. baumannii, Pseudomonas aeruginosa PA14, E. coli, S. aureus, B. subtilis and C. albicans were examined by determining the growth curves and viability of the microorganisms. Since Cu₂O is a photoactive material, the experiments were conducted in dark conditions. To determine the growth curves for the different species of bacteria and the fungus, 100 μL of polysaccharide (0.7% w/v), Cu₂O-polysaccharide complex (0.7% w/v polysaccharide with 500 ppm of copper ions) or Cu₂O (500 ppm copper ions) solution were mixed with 900 μL of suitable medium and 10 μL of microbial culture at OD=1. A sample of 200 μL of each combination was incubated with shaking in 96-well plates at 37° C. for 14 h for A. baumannii, Pseudomonas aeruginosa PA14, E. coli, S. aureus and B. subtilis or 48 hr. for C. albicans. The turbidity of the medium was measured hourly with a micro-plate reader (BioTek Instruments) at a wavelength of 600 nm. Growth inhibition was calculated from the following formula:

$$Inhibitory index ( \% ) = ( 1 - \frac{{OD}_{treatment} - {OD}_{treatment \_ blank}}{{OD}_{control} - {OD}_{control \_ blank}} ) \times 100 \qquad ( Equation 0 )$$

where ODₜᵣₑₐₜₘₑₙₜ is the absorbance of the sample of Cu₂O-polysaccharide complex or polysaccharide plus bacteria at t=14 hr. (in the logarithmic phase of growth) or fungus at t=48 hr., OD_{treatment_blank} is the absorbance of the same sample without bacteria or fungus at the same time, OD_{control} is the absorbance of only LB or TSB (as mentioned above for each bacteria) and bacteria or PDB and fungus, and OD_{control_blank} is the absorbance the same sample without bacteria or fungus at the same time. For cell viability determination, cells were incubated in test tubes containing 900 μL of LB, TSB or PDB, as relevant, mixed with 100 μL of Cu₂O-polysaccharide solution (0.7% w/v polysaccharide with 500 ppm copper ions) vs their respective controls (LB, TSB or PDB alone, or solutions of polysaccharide or copper ions). After 24 hr., 100 μL of each sample was plated on an LB, TSB or PDB agar plate after serial dilution and incubated overnight at 37° C. (under dark conditions). On the following morning, CFUs were counted.

Biofilm Characterization: The morphological properties of the coatings and the biofilm structure were analyzed using SEM. Imaged biofilms of Pseudomonas aeruginosa PA14 and C. albicans on Cu₂O-polysaccharide, polysaccharide and Cu₂O surfaces were acquired using high-resolution (HR)-SEM. After 24 hr. of incubation, the samples were prepared for SEM studies as follows. After fixation in 2.5% buffered glutaraldehyde, the samples were subsequently dehydrated via an increasing serial ethanol gradient and immersed in a hexamethyldisilazane (HMDS)/ethanol gradient solution (25%, 50%, 75%, 90%, 95% and 100%). The treated specimens were air dried for 4 hr., and in preparation for SEM scanning (JSM-7400F, JEOL) they were sputter coated with a 20 nm layer of gold using an EMITECH K575x sputtering device (Emitech Ltd, UK).

Statistical Analysis: Means±deviations (SD) from triplicate end point data were presented in all tables. Differences between the control and samples were determined using one-way analysis of variance (ANOVA) and the Tuckey post hoc test with GraphPad Prism 6 for Windows software. Statistically significant differences were set at p<0.05.

### Example 1

### Chemical and Rheological Characterization of the Cu₂O-Polysaccharide Complex

The first step in studying the complexation of the Porphyridium sp. polysaccharide with Cu₂O (pl=7.5) was to characterize Cu₂O-polysaccharide complexes differing in their Cu₂O contents by chemical and physical approaches. For purposes of this specification and the accompanying claims, the term “pl” is the pH at which a molecule carries no net electrical charge or is electrically neutral in the statistical mean.

Table 1 (presented below) summarizes the relationship between the concentration of added Cu [from 0 ppm (native polysaccharide) to 750 ppm] on the viscosity, conductivity and zeta potential of the Cu₂O-polysaccharide complexes.

All three parameters increased with increasing Cu concentrations, with the viscosity and conductivity rising markedly and the zeta potential less so. The results showed that the native polysaccharide and the Cu₂O-polysaccharide complexes exhibited similar values of the zeta potential (range of −45 to −49 mV). The zeta potential of the Cu₂O solutions (250/500/750 ppm Cu in buffer, pH 4.5) ranged from +20.5 to +22.9.

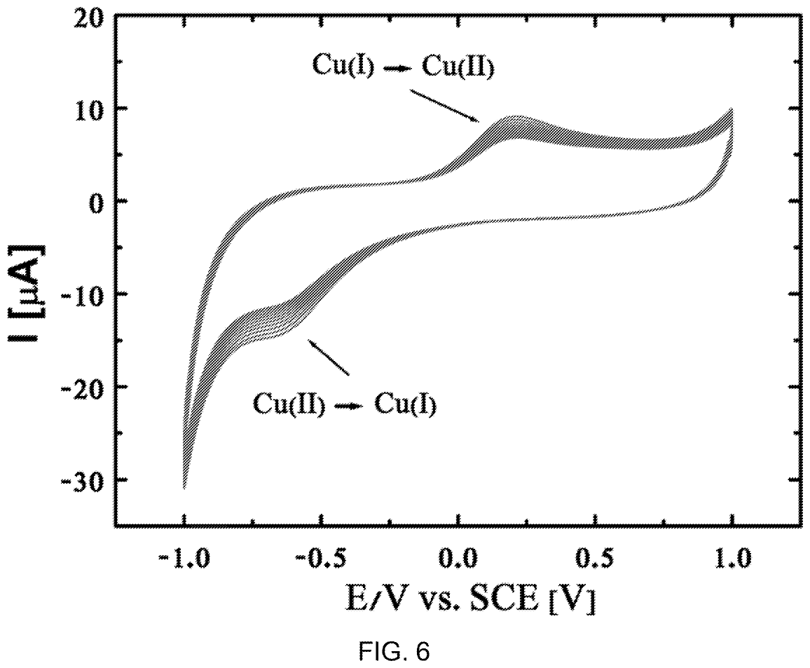

In addition, cyclic voltammetry analysis (FIG. 6) of the Cu₂O-polysaccharide complex showed that the oxidation state of copper does not change while in the complex.

FIG. 6 is a cyclic voltammogram {l[μA] as a function of (E/V vs SCE [V])} of Cu₂O-PS complex (0.7% (w/v) PS and 500 ppm copper) in acetonitrile: GCE working, Ag/AgCl reference, and Pt auxiliary electrodes and lithium perchlorate as a supporting electrolyte; [lithium perchlorate]=2 M.

Since all three Cu₂O-polysaccharide complexes exhibited higher viscosities and conductivities than the native polysaccharide and similar zeta potentials to that of the native polysaccharide, it was decided to further develop and characterize the complex containing 500ppm copper and 0.7% of the polysaccharide.

TABLE 1
Effect of the Addition of Copper on the Viscosity, Conductibity
and Zeta Potential of Cu₂O-Polysaccharide Complexes.

| | copper | | con- | zeta |
|---|---|---|---|---|
| | added | viscosity | ductivity | potential |
| Sample | (ppm) | (cP)* | (μs)* | (mV)* |
| Polysaccharide** | — | 1.830 ± 125 | 1.150 ± 20 | −45 ± 3 |
| Cu₂O- | 250 | 1.972 ± 220 | 2.532 ± 44 | −47 ± 2 |
| polysaccharide | 500 | 2.532 ± 110 | 2.857 ± 15 | −48 ± 0 |
| | 750 | 2.328 ± 124 | 3.235 ± 20 | −49 ± 3 |

The Cu₂O-polysaccharide complex was prepared by directly adding Cu₂O to the polysaccharide and stirring the mixture gently with a magnetic stirrer for 24 h at room temperature.
*Values are means ± SD of three different experiments performed in triplicate.
**Polysaccharide concentration in all samples was 0.7% (w/v) and the pH was 4.5.

The Cu₂O-polysaccharide complex was prepared by directly adding Cu₂O to the polysaccharide and stirring the mixture gently with a magnetic stirrer for 24 h at room temperature.

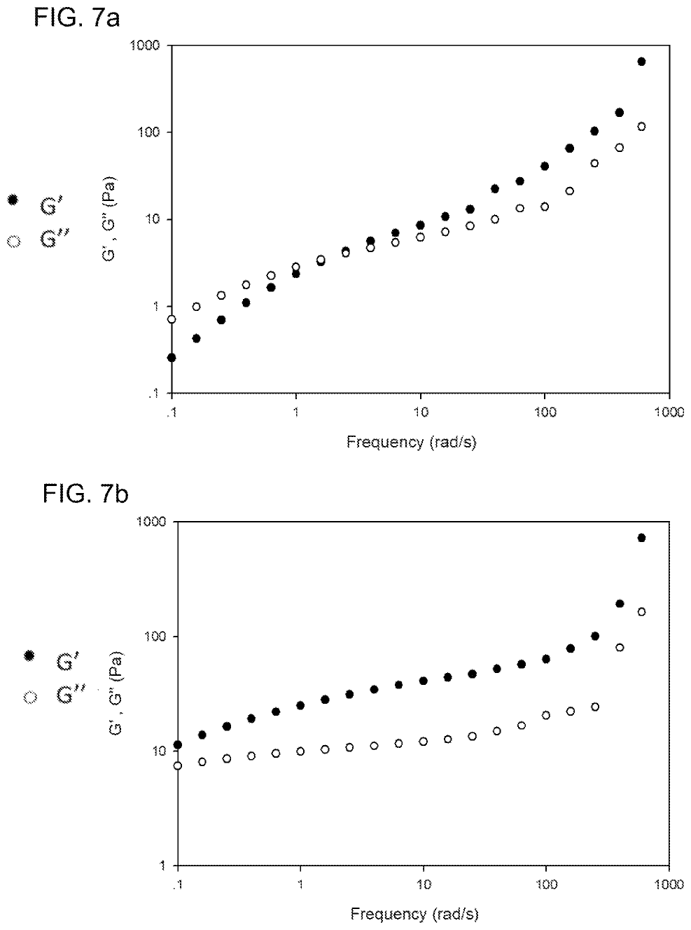

The dynamic viscoelastic properties of the polysaccharide and the Cu₂O-polysaccharide complex was characterized in terms of the frequency dependence of the storage modulus G′(ω) and the loss modulus G″(ω) (FIG. 7a and FIG. 7b).

FIG. 7a is a mechanical spectrum of PS (0.7% w/v) indicating G′ (storage modulus) and G″ (loss modulus) as a function of angular frequency (ω) at 25° C. and FIG. 7b is a mechanical spectra of Cu₂O-PS complex (0.7% w/v PS and 500 ppm copper) indicating G′ (storage modulus) and G″ (loss modulus) as a function of angular frequency (w) at 25° C.

Results presented in FIG. 7a and FIG. 7b suggest weak gel behavior only for the polysaccharide solution (but not for solutions of the complex), in agreement with previous studies showing that a 1% (w/v) solution of polysaccharide behaves as a weak gel. The shift of the crossover position in the Cu₂O-polysaccharide complex to lower than the measurable range (<0.1 rad/s) indicated a longer relaxation time of the chains and demonstrated that the solution behavior of the complex resembled that of a soft gel.

### Example 2

### Spectrophotometric and Microscopic Analysis of the Cu₂O-Polysaccharide Complex

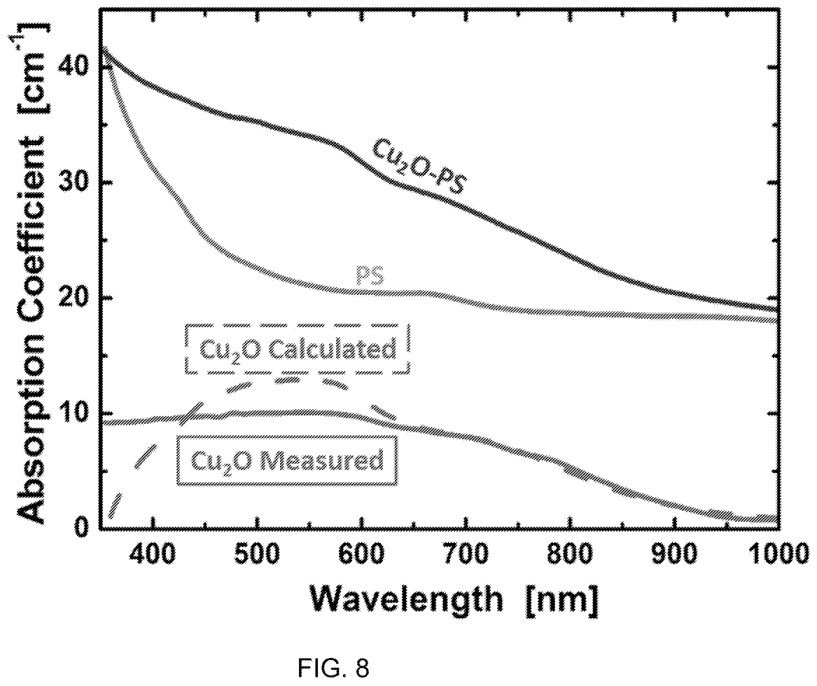

Absorption coefficient spectra were used to determine whether there had indeed been a reaction between the polysaccharide and Cu₂O. To this end, spectra of the absorption coefficients derived from the transmittance and reflectance spectra of the Cu₂O-polysaccharide complex were compared to the spectra of the unmixed constituents, i.e., polysaccharide and Cu₂O (FIG. 8).

FIG. 8 is a plot of absorption coefficient [cm⁻¹] as a function of wavelength [nm] for PS (0.7% w/v; green line), Cu₂O-PS complex (0.7% w/v PS; 500 PPM copper; red line), and Cu₂O alone (500 ppm measured and calculated, solid and dashed blue lines, respectively); dashed blue line shows the Cu₂O curve calculated from the polysaccharide and Cu₂O-polysaccharide curves using Eq. 2 (below).

Spectra were obtained using Newport Corp. 2931 power meter with a Si detector. The sample was illuminated using halogen (for IR range) and Xe short-arc (for visible and UV range) light sources. The light was monochromatized using a Newport Corp. MS257 monochromator equipped with long-pass order-sorting filters. The spectrometer was operated in a closed control loop to maintain a constant photon flux throughout the spectral range of the measurement.

The absorption coefficient for each material was calculated from measured transmittance and reflectance spectra using the Beer-Lambert law for solids:

$$α = \frac{1}{t} ln ( \frac{{( 1 - R )}^{2}}{T} ) \qquad ( Equation 1 )$$

where α is the absorption coefficient, t is the thickness of the layer of the studied material, R—measured reflectance, and T—measured transmittance.

The plots for the polysaccharide and the Cu₂O-polysaccharide complex appeared to be significantly different, giving rise to the question of whether the absorption exhibited by the Cu₂O-polysaccharide complex could indeed be attributed to a new compound or whether it should be attributed to a non-reacted mixture of the components (which would have given a plain superposition of their absorption coefficient spectra). To answer this question, we performed the following calculation:

$${α}_{PS + {Cu}_{2} O} \approx {α}_{PS} + {α}_{{Cu}_{2} O} = \frac{1}{t} ln ( \frac{{( 1 - {R}_{PS} )}^{2} {( 1 - {R}_{{Cu}_{2} O} )}^{2}}{{T}_{PS} {T}_{{Cu}_{2} O}} ) \qquad ( Equation 2 )$$

To obtain the values for the above equation, the absorption coefficient of Cu₂O was first calculated by subtracting the absorption coefficient of the polysaccharide from that of Cu₂O-polysaccharide complex. The resulting plot is shown as a blue dashed line in FIG. 8. Clearly, the calculated curve for Cu₂O is significantly different from the curve derived from the measured Cu₂O spectrum over wavelengths shorter than 650 nm. This comparison suggests that a reaction had indeed taken place between the polysaccharide and Cu₂O, at least partially.

The difference between the measured and calculated Cu₂O curves suggests that a reaction between PS and Cu₂O takes place, at least partially.

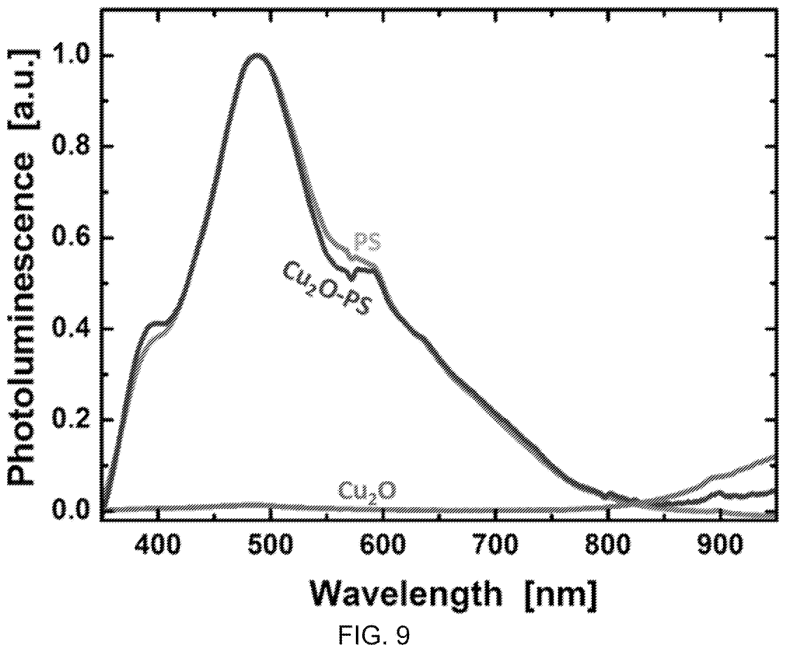

Similarly, the luminescence behavior of solutions of the Cu₂O-polysaccharide complex and the polysaccharide were significantly different from that of the Cu₂O solution, with the latter solution exhibiting low luminescence intensity (FIG. 9).

FIG. 9 is a plot of Photoluminescence [a.u.] as a function of wavelength [nm] for PS 0.7% (w/v) (green), Cu₂O-PS complex (0.7% (w/v) polysaccharide and 500 ppm copper) (red) and Cu₂O 500 ppm (blue) showing that addition of Cu₂O to PS increased photoluminescence at wavelengths >800 nm; photoluminescence spectra were acquired on a Newport Corp. MS257spectrometer equipped with a Si CCD detector and long-pass order-sorting filters and a 1-mW He-Cd laser was used for excitation;

The main difference between the spectra of the polysaccharide and the Cu₂O-polysaccharide complex was observed in the infra-red range of the spectrum, manifested as a rise in the photoluminescence intensity of the complex at wavelengths longer than 800 nm. This rise may be attributed to the contribution of the added Cu₂O, since a similar rise was also observed in the photoluminescence spectrum of the Cu₂O alone.

To elucidate how the copper binds to the polysaccharide, FT-IR spectroscopy was used.

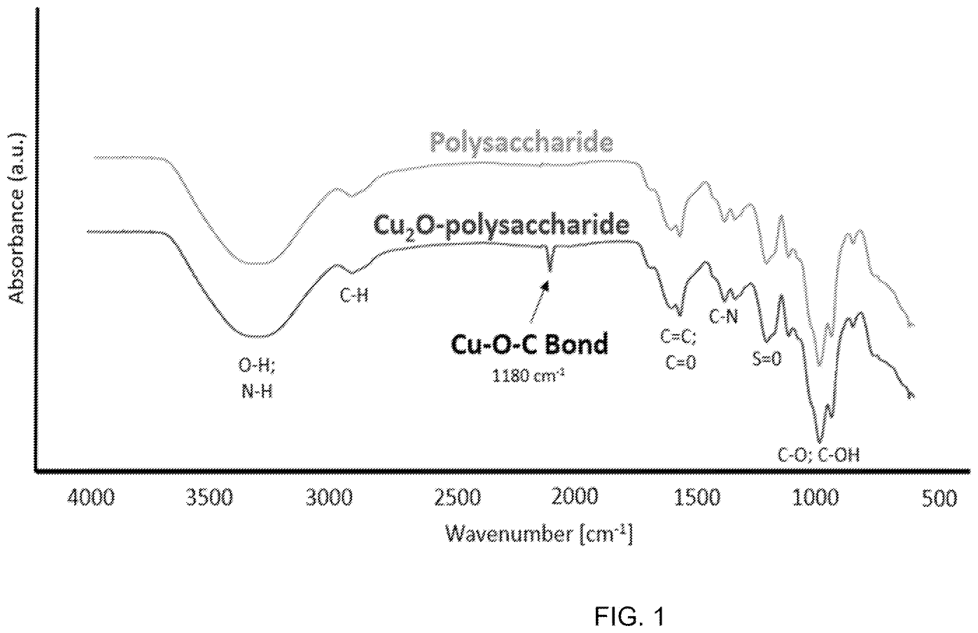

FIG. 1 is a plot of FT-IR the transmission spectra (absorbance units (a.u.) as a function of wavelength (cm⁻¹)) of polysaccharide (PS) and the Cu₂O-PS complex (0.7% polysaccharide (w/v) and 500 ppm copper) in the region of 650-4000 cm⁻¹;

The transmission spectrum of the Cu₂O-polysaccharide complex differed from that of the polysaccharide in that the former exhibited a new peak at 1180 cm⁻¹, suggesting the formation of a new covalent bond. In both spectra (Cu₂O-polysaccharide complex and polysaccharide), the broad band centered at 3260 cm⁻¹ was assigned to O—H stretching vibrations, and the weak signal at 2926 cm⁻¹, to C—H stretching vibrations (Gómez-Ordóñez et al., (2011) Food Hydrocoll. 25 (6):1514-1520.) Both the polysaccharide and the Cu₂O-polysaccharide complex also exhibited a broad band around 1220-1260 cm⁻¹, which was assigned to sulfated ester groups (S═O)—also a characteristic component of the sulfated polysaccharides of other microalgae and indeed of other algae, such as alginate in brown seaweeds.

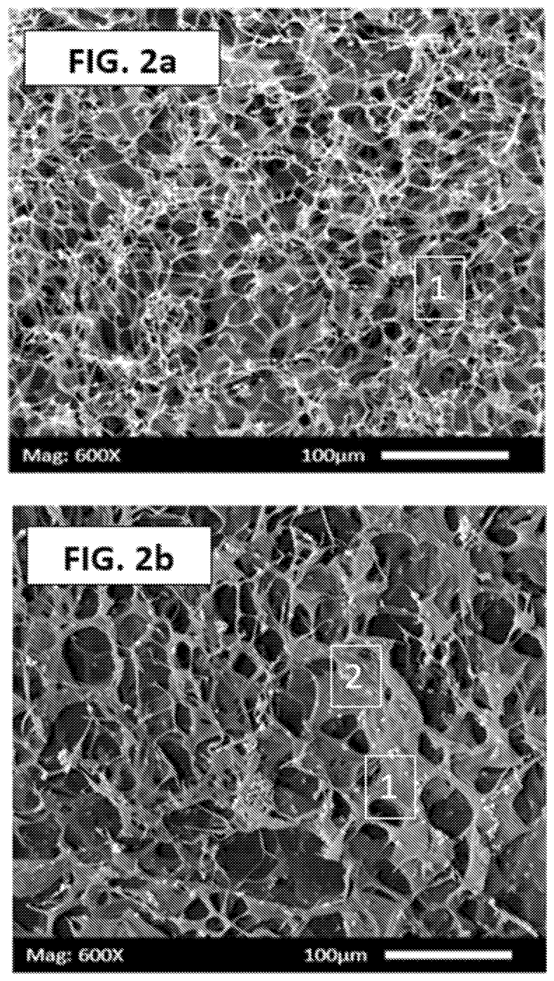

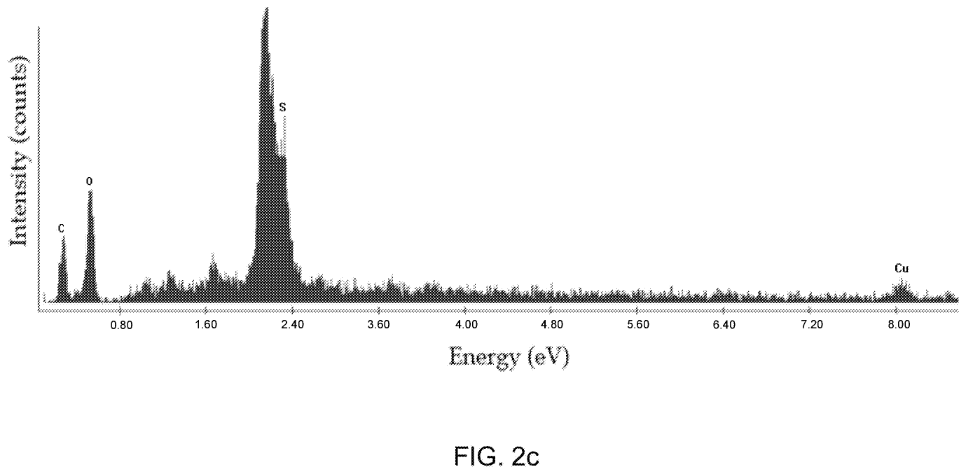

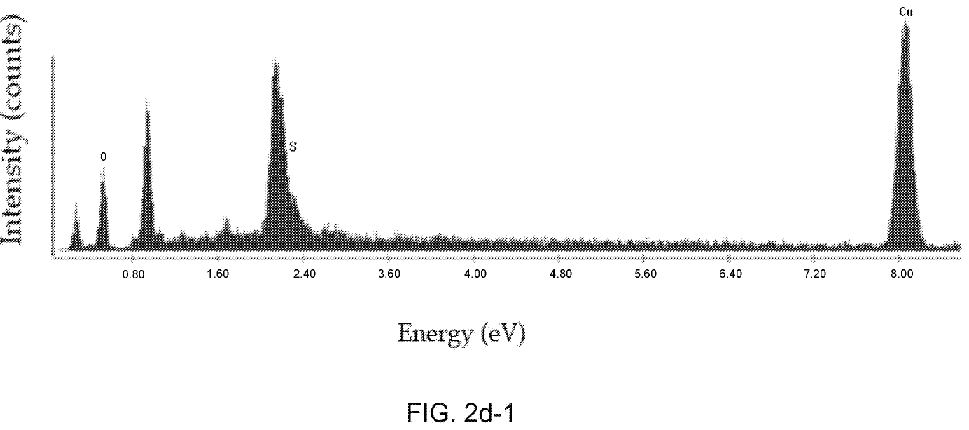

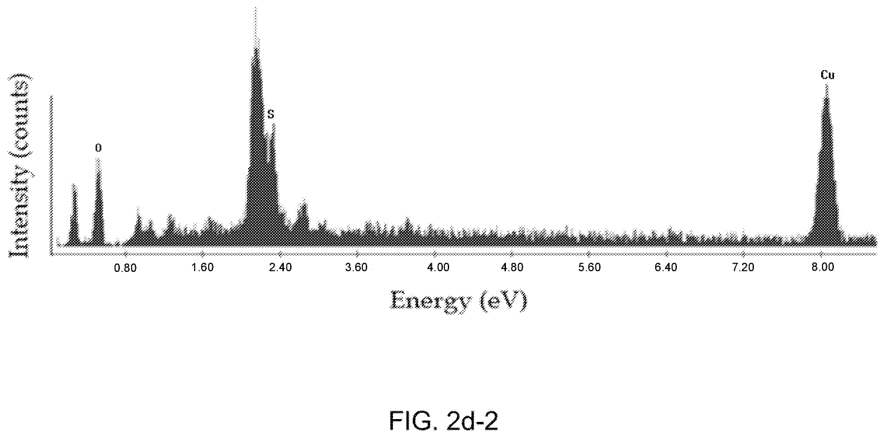

The surface morphologies of the polysaccharide and the Cu₂O-polysaccharide complex were studied by SEM (FIG. 2a and FIG. 2b).

FIG. 2a is an SEM micrograph (600×) of 0.7% PS (w/v) alone and (B) the Cu₂O-polysaccharide complex and FIG. 2b is an SEM micrograph (600×) of Cu₂O-PS complex in 0.7% PS (w/v) at a copper concentration of 500 PPM.

FIG. 2a and FIG. 2b show that the structure of the polysaccharide is porous and fibrous. Differences can be seen in the quantity and in the size of the pores between the polysaccharide and the Cu₂O-polysaccharide complex, with the complex having fewer entanglements and larger pores. In addition, in corroboration with the AFM results (see below), the Cu₂O-polysaccharide complex showed a distinct morphology with bright, uniformly scattered needle-like structures (spikes). EDS-used to analyze the chemical compositions of the Cu₂O-polysaccharide complex and the polysaccharide-showed five peaks corresponding to five major elements (FIG. 2c, FIG. 2d-1 and FIG. 2d-2)), with the dominant gold peak being due to the gold coating.

FIG. 2c is an EDS spectrum (intensity counts as a function of energy (eV)) of the polysaccharide sample of FIG. 2a (area 1);

FIG. 2d-1 is an EDS spectrum (intensity counts as a function of energy (eV)) of the Cu₂O-PS complex sample of FIG. 2b in a smooth area (area 1) and FIG. 2d-2 is an EDS spectra (intensity counts as a function of energy (eV)) of the Cu₂O-PS complex sample of FIG. 2b in a spiked area (area 2).

The main elements identified in the polysaccharide sample were carbon [61.87% (w/v)] and oxygen [30.87% (w/v)], as is to be expected for a sugar-containing polymer. In addition, sulfur [5.57% (w/v)] and small traces of copper [1.69% (w/v)], probably originating from the growth medium (which contains microelements including copper), were also evident. FIG. 2d-1 and FIG. 2d-2 show the elemental composition of Cu₂O-polysaccharide complex detected by EDS (1—of the smooth area, and 2—of the grainy area, i.e., the spikes, as seen in FIG. 2b). FIG. 2d-1 shows the elemental composition of Cu₂O-polysaccharide complex detected by EDS. Again, the main elements identified in this sample were carbon [48.86% (w/v)] and oxygen [31.95% (w/v)] and small amount of sulfur [8.06% (w/v)]. The main elements identified in the spikes (FIG. 2d-2) were carbon [46.44% (w/v)] and oxygen [26.78% (w/v)] and a small amount of sulfur [12.53% (w/v)] indicating that the spikes were derived from the polysaccharide. As expected, the quantity of copper in the complex [11.13-14.24% (w/v)] was significantly higher than that in the polysaccharide. It is assumed that the Cu is located on the surface of the complex.

### Example 3

### Atomic Force Microscopy (AFM)

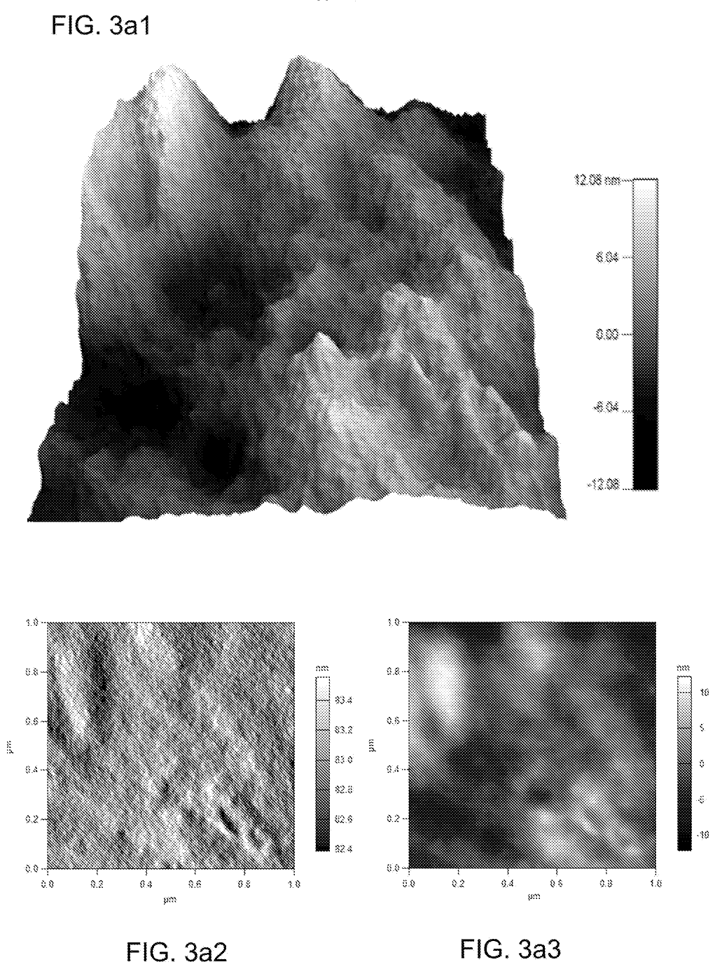

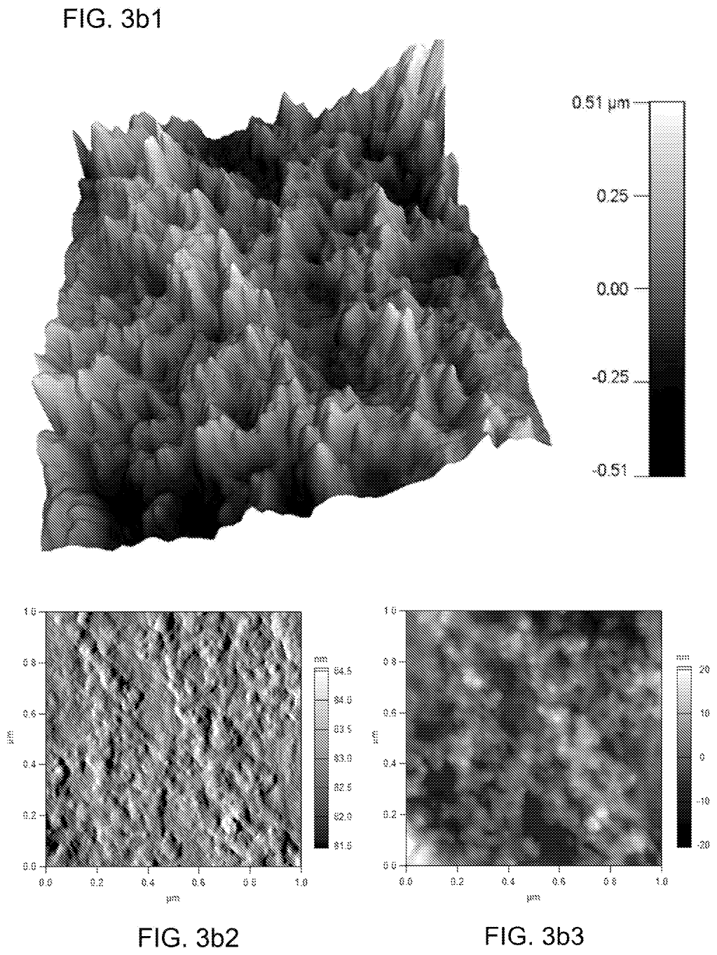

In order to further study the surface topography and morphology of the Cu₂O-polysaccharide complex, AFM was used (FIG. 3a1, FIG. 3a2, FIG. 3a3, FIG. 3b1, FIG. 3b2, and FIG. 3b3).

FIG. 3a1 is an AFM surface topography and 3D image of 0.7% (w/v) PS and showing smooth surface of the polysaccharide

FIG. 3a2 is 2D SEM image of 0.7% (w/v) PS.

FIG. 3a3 is 2D AFM image of 0.7% (w/v) PS.

FIG. 3b1 is an AFM surface topography and 3D image of Cu₂O-PS complex (0.7% (w/v) PS and 500 ppm copper) showing needle-like structures of the complex

FIG. 3b2 is 2D SEM image Cu₂O-PS complex (0.7% (w/v) PS and 500 ppm copper).

FIG. 3b3 is 2D AFM image Cu₂O-PS complex (0.7% (w/v) PS and 500 ppm copper).

The polysaccharide sample exhibited a generally smooth surface, but with some roughness throughout, i.e., the raised structures were approximately 24.16-40.5 nm high and 1-10 nm wide. In contrast, the surface of the Cu₂O-polysaccharide complex was characterized by needle-like structures-spikes-that protruded up to 1,000 nm above the surface and had a thickness of approximately 10-20 nm (FIG. 3b1). The above morphologies were similar to those previously described for polysaccharide and Cu-polysaccharide complexes. Using both Gwyddion and ImageJ software the number of the spikes on the Cu₂O-polysaccharide complex surface was counted and found to be about 5,000±400 spikes/μm².

### Example 4

### Antimicrobial Activity of the Cu₂O-Polysaccharide Complex

The antimicrobial activity of the Cu₂O-polysaccharide complex was evaluated (using turbidity measurements) in various model microbial pathogens, namely, the fungus C. albicans (generation time 82 min), the Gram-negative bacteria A. baumannii (generation time 48 min), Pseudomonas aeruginosa PA14 (generation time 35 min) and E. coli (generation time 21 min), and the Gram-positive bacteria, S. aureus (generation time 30 min) and B. subtilis (generation time 28 min) (Table 2; presented below). Because of the dilution process of the assay (as described in the Experimental section), the final concentration of copper was 30 ppm, and polysaccharide concentration was 0.07% (w/v). The Cu₂O-polysaccharide complex almost completely inhibited the growth of C. albicans (91.7±0.4% inhibition as compared with untreated cells), whereas Cu₂O alone exhibited low activity (about 15% inhibition).

The polysaccharide alone exhibited moderate activity in inhibiting C. albicans (35.8±1.2% inhibition).

For the various bacterial species, the Cu₂O-polysaccharide complex exhibited 72-78% inhibition. Cu₂O alone did not inhibit bacterial growth, and the polysaccharide alone demonstrated only moderate inhibition (reduction of about 30-49% in growth for the bacteria, respectively, as compared with untreated cells).

TABLE 2
Effect of Cu2O-Polysaccharide Complex on the Inhibition of Growth of
C. albicans, A. baumannii, P. aeruginosa, E. coli, S. aureus and B. subtilis

| | | % inhibition with: | | |
|---|---|---|---|---|
| time of | | | | Cu₂O- |
| exposure | microorganism | polysaccharide | Cu₂O | Polysaccharide |
| 48 h | C. albicans | 35.8 ± 1.2 | 14.7 ± 1.9 | 91.7 ± 0.4 |
| 14 h | A. baumannii | 40.1 ± 3.5 | 9.7 ± 2.9 | 78.5 ± 9.8 |
| | P. aeruginosa | 45.9 ± 0.9 | 4.9 ± 6.5 | 78.1 ± 5.1 |
| | E. coli | 49.2 ± 4.1 | 1.6 ± 1.8 | 75.0 ± 1.5 |
| | S. aureus | 30.8 ± 5.6 | 20.9 ± 8.9 | 72.9 ± 7.1 |
| | B. subtilis | 29.6 ± 2.9 | 15.6 ± 2.7 | 79.7 ± 2.5 |

The Cu₂O-polysaccharide complex contained 0.07% (w/v) polysaccharide and 30 ppm copper.

Values in the tables are means±SD of three different experiments performed in triplicate.

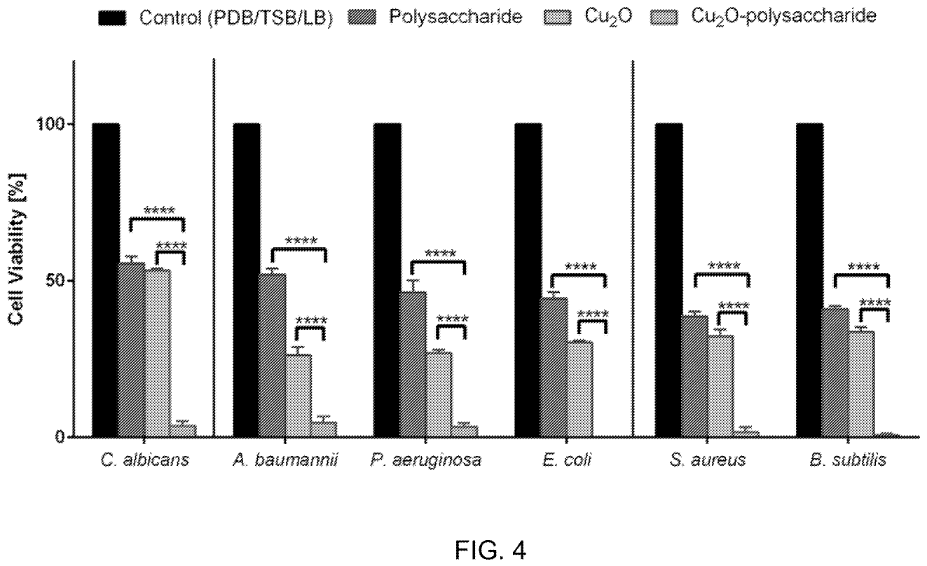

To investigate the bactericidal effect of the Cu₂O-polysaccharide complex, the viability of the fungal and bacterial cells was determined in a colony forming units (CFU) assay. Cells were inoculated on agar plates and allowed to grow at 37° C. After an overnight incubation with the Cu₂O-polysaccharide complex or its controls (growth medium alone or polysaccharide or Cu₂O solutions alone), CFUs were counted, and cell viability was determined in comparison with the control values (growth medium plus polysaccharide or Cu₂O). The results show that the most effective treatment against C. albicans was that of the Cu₂O-polysaccharide complex (FIG. 4), with about 3% viable cells after the treatment as compared with ˜45% viable cells with polysaccharide alone or the copper oxide alone.

FIG. 4 is a bar graph of % viability C. albicans, A. baumannii, Pseudomonas aeruginosa, E. coli, S. aureus and B. subtilis cultures exposed to no treatment (black bar), 0.07% (w/v) PS (green bar), Cu₂O solution (30 PPM copper) (blue bar) and Cu₂O/PS complex solution [0.07% (w/v) PS and 30 PPM copper] with **** indicating a significant difference (p<0.05) by ANOVA test.

For all bacteria, no or minimal bacterial growth was detected. With the Cu₂O-PS complex. Thus, the Cu₂O-polysaccharide complex not only inhibited fungal and bacterial growth, it also caused cell death.

In summary, the antifungal and antibacterial activities of the Cu₂O-polysaccharide complex are more potent than those of the polysaccharide or Cu₂O alone. The viability test also showed that the Cu₂O-polysaccharide complex not only inhibited microbial growth but also reduced the number of viable cells. The strongest effect was obtained for the Cu₂O-polysaccharide complex, which caused 91% inhibition of C. albicans growth after 48 h and 97% cell death after 24 h. It seems that the antimicrobial potency of the Cu₂O-polysaccharide complex vs. the polysaccharide or Cu₂O alone is probably due to the spikes on the surface of the Cu₂O-polysaccharide complex. The results of Example 3 suggest that the observed nano-spikes may play a role in the antifungal/antibacterial effect.

### Example 5

### Anti-Biofilm Activity

In order to investigate the anti-biofilm activity of the Cu₂O-polysaccharide complex, C. albicans and Pseudomonas aeruginosa PA14, used as model pathogens, were grown on clean glass surfaces in the presence and absence (as the control) of the Cu₂O-polysaccharide complex or the polysaccharide or Cu₂O alone.

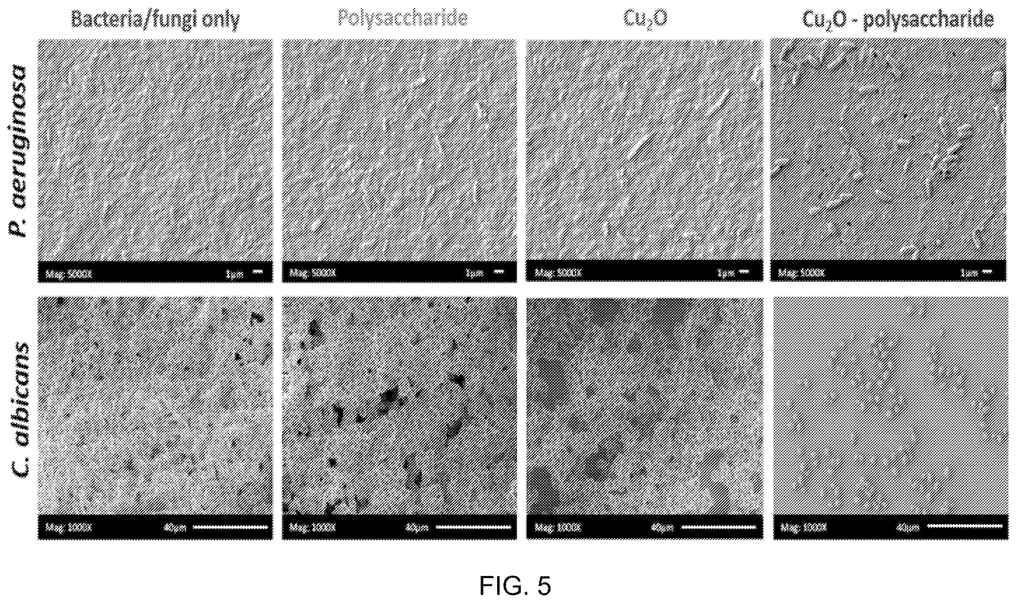

FIG. 5 is series of HR-SEM images of biofilm assays against C. albicans and Pseudomonas aeruginosa PA14 using clean glass surface (leftmost image in each row), PS only (0.7% w/v); Cu₂O (500 ppm) only and Cu₂O-PS complex (0.7% (w/v) PS and 500 ppm copper).

The Cu₂O-polysaccharide complex (dehydrated) on the glass surface demonstrated significantly improved microbial clearance as compared to a polysaccharide film and Cu₂O alone (FIG. 5). In response to the Cu₂O-polysaccharide complex treatment, only a few individual cells were observed. Anti-biofilm activity was less evident on the surface treated with the polysaccharide or Cu₂O alone (FIG. 5).

### Example 6

### Comparison to Bifonazole

In order to understand the relative efficacy of a Cu₂O-PS complex as a potential topical antifungal, it was tested in comparison to Bifonazole.

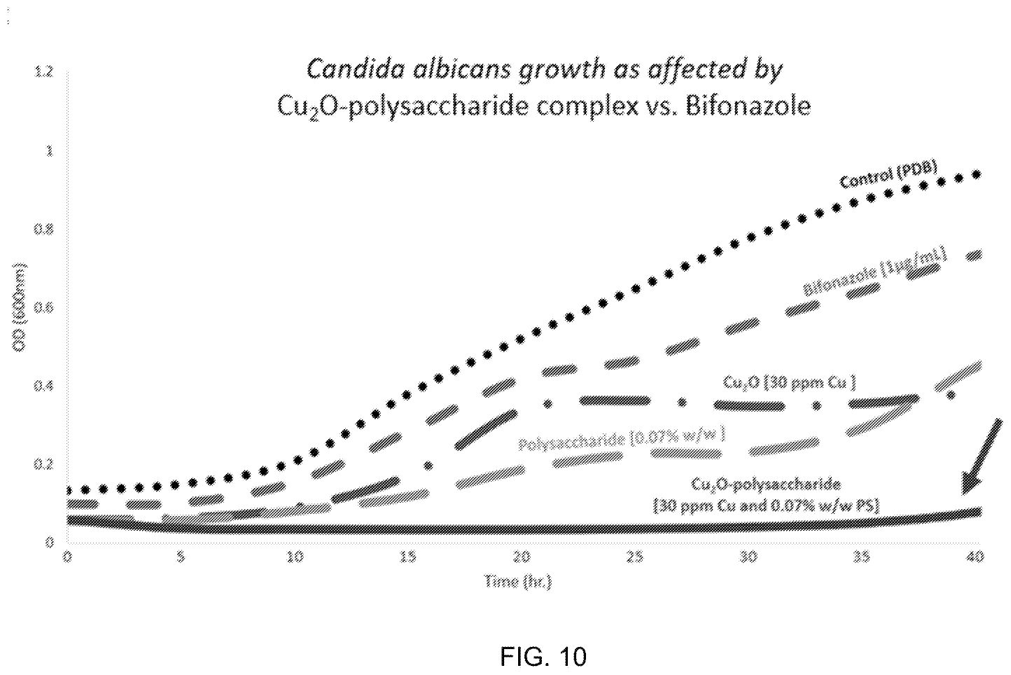

FIG. 10 is a plot of OD (600 nm) as a function of time (hr) for Candida albicans cultures exposed to Bifonazole (1 μg/mL; blue line), Cu₂O (30 ppm Cu; purple line), PS (0.07% w/v; green line), Cu₂O-PS complex (0.07% w/v PS with 30 ppm Cu; red line) and negative control culture (black line).

Results presented in FIG. 10 show that the Cu₂O-PS complex was superior to all other tested compounds both in terms of strength of initial effect and in terms of duration of effect.

### Example 7

### Survey of Monovalent and Divalent Copper Complexes with Polysaccharide

In order to investigate how other sources of copper behave when complexed to polysaccharide four Monovalent (Cu₂O and CuCl) and Divalent (CuO and CuCl₂) Cu-PS complexes were tested in a leaching assay.

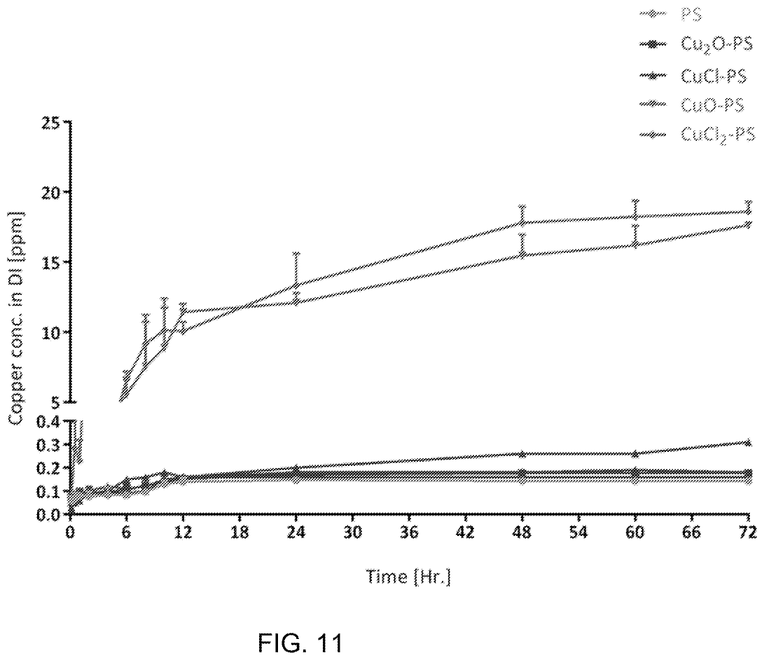

FIG. 11 is a plot of copper concentration in DI (ppm) as a function of time (hr.) for Monovalent and Divalent Cu-PS complexes to DI medium: PS only (green line), Cu₂O-PS (red line with squares), CuCl-PS (red line with triangles), CuO-PS (blue line with triangles), CuCl₂-PS (blue line with diamonds) In this experiment, all Cu-PS complexes contain 0.7% (w/v) polysaccharide and 500 ppm copper.

Results presented in FIG. 11 indicate that Divalent Cu-complexes exhibited 3% leaching and Monovalent Cu-complexes exhibited 0.04% leaching.

The antimicrobial activity of the Cu-PS complexes is presented in Table 3. As can be seen in Table 3 (upper portion) the growth inhibition of the Monovalent Cu-complexes was significantly higher than that of the Divalent Cu-complexes in both fungi and bacteria. The Cu-monovalent complexes almost completely inhibited the growth of C. albicans (93 and 89% inhibition for Cu₂O-PS and CuCl-PS, respectively, as compared with untreated cells), whereas Cu-Divalent complexes showed, and the polysaccharide alone exhibited moderate activity in inhibiting C. albicans. For all bacterial species, the Cu-Monovalent complexes exhibited 75-83% inhibition. The antibacterial activity of the Cu-Monovalent complexes was higher than that of the Cu-Divalent copper complexes. And copper salts alone did not inhibit fungal and bacterial growth as compared with untreated cells.

To investigate the lytic effect of the Cu-PS complexes, the cell viability of the fungal and bacterial cells was determined in a CFU assay. Cells were inoculated for 24 hours on agar plates and allowed to grow at 37° C. (Table 3 lower portion). After an overnight incubation with the Cu-PS complexes or its controls (growth medium alone or polysaccharide or copper solutions alone), CFUs were counted, and cell viability was determined in comparison with the control values (containing growth medium and bacteria/fungi only). The results show that the most effective treatment against C. albicans was that of the Cu-Monovalent complexes (Table 3b), with about 5-12% viable cells after the treatment as compared with 55-90% viable cells with polysaccharide alone or the copper alone. The Cu-Divalent copper complexes shown moderate activity against all pathogens (˜21-42% viability). Similarly, low cell viabilities were previously reported for Cu₂O. For all bacteria, no or minimal bacterial growth was detected. Thus, it seems that the Cu₂O-polysaccharide complex not only inhibited fungal and bacterial growth but also caused cell death. A few studies have reported on the activity of Cu₂O against bacteria and fungi. One of those studies showed relatively high antifungal activity of Cu₂O-Cu nanoparticles/alginate (30 ppm Cu) against Neoscytalidium dimidiatum, but only after a long incubation period of 8 days. In a different study, an additive effect was found for chitosan copper against Candida albicans, Candida parapsilosis, E. coli, and P. aeruginosa when higher copper concentrations were used (>1000 ppm). Relatively high activity was also observed with PET/Cu₂O@ZrP nanosheets having a high Cu content (186,200 ppm) against S. aureus, E. coli, and C. albicans.

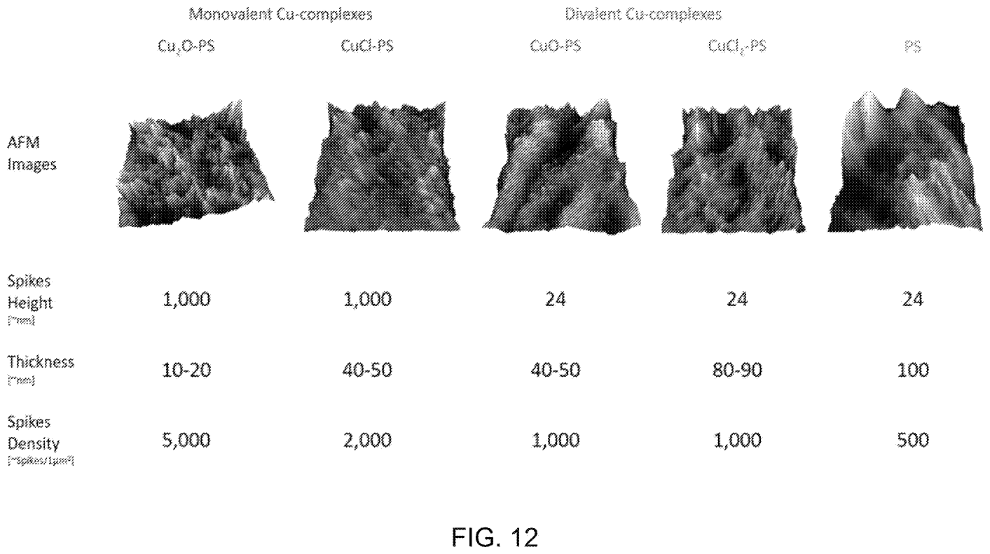

In our previous study we have shown that the Cu₂O-PS complex have significantly higher and more dense spikes. In FIG. 12 the AFM topography of the complexes are presented. Spikes maximal height of the Monovalent Cu-complexes are much higher than the Divalent Cu-complexes (1,000 nm and 24 nm, respectively) whereas the Monovalent Cu₂O-PS complex have sharper spikes as compared with the Monovalent CuCl-PS complex, the Divalent Cu-complexes and the PS alone. Spikes density is also higher in the Monovalent Cu-complexes, the Monovalent Cu-complex with Cu₂O being higher than that of the CuCl-PS complex. It is worth noting here that the Monovalent and Divalent copper chloride complexes are thicker than their copper oxide counterparts.

TABLE 3
Effect of the Mono and Divalent Cu-complexes on the (a) Inhibition of Growth
and (b) cell viability of Fungi, Gram-Negative and Gram-Positive Bacteria

| | Microorganism | | | | | |
|---|---|---|---|---|---|---|
| Test | C. | A. | P. | E. | S. | B. |
| Compound | albicans | baumannii | aeruginosa | coli | aureus | subtilis |

(a) Growth Inhibition [% from control]

| | | | | | | |
|---|---|---|---|---|---|---|
| Cu₂O—PS | 92.8 ± 0.1 | 78.5 ± 9.8 | 78.1 ± 5.1 | 75.0 ± 1.5 | 72.9 ± 7.1 | 79.3 ± 2.5 |
| CuCl—PS | 88.9 ± 2.3 | 69.4 ± 2.7 | 66.7 ± 2.3 | 75.8 ± 2.9 | 75.9 ± 3.2 | 83.4 ± 5.9 |
| CuO—PS | 21.2 ± 5.7 | 48.4 ± 5.9 | 40.2 ± 6.0 | 59.1 ± 5.3 | 18.9. ± 32 | 15.7 ± 4.2 |
| CuCl₂—PS | 48.7 ± 0.1 | 45.9 ± 4.1 | 44.8 ± 3.5 | 66.4 ± 1.2 | 14.3 ± 8.9 | 15.3 ± 2.0 |
| PS | 12.2 ± 1.7 | 32.4 ± 3.6 | 38.6 ± 8.3 | 49.2 ± 4.1 | 30.8 ± 5.6 | 29.6 ± 2.9 |
| Cu₂O | 4.7 ± 0.1 | 22.4 ± 4.9 | 21.5 ± 9.4 | 15.2 ± 2.0 | 20.9 ± 8.9 | 10.6 ± 5.8 |
| CuCl | 2.3 ± 1.6 | 5.6 ± 3.5 | 4.1 ± 4.2 | 7.8 ± 2.9 | 6.5 ± 3.4 | 10.5 ± 4.5 |
| CuO | 2.5 ± 0.3 | 4.5 ± 5.6 | 9.7 ± 2.8 | 1.6 ± 0.9 | 9.8 ± 4.8 | 8.5 ± 2.5 |
| CuCl₂ | 1.8 ± 0.7 | 7.8 ± 6.5 | 7.8 ± 4.1 | 4.3 ± 2.0 | 8.9 ± 5.2 | 9.6 ± 5.6 |

(b) Cell Viability [%]

| | | | | | | |
|---|---|---|---|---|---|---|
| Cu₂O—PS | 5.6 ± 0.5 | 4.6 ± 0.8 | 3.6 ± 1.3 | 3.8 ± 2.3 | 2.0 ± 0.3 | 1.0 ± 0.9 |
| CuCl—PS | 11.7 ± 0.6 | 6.7 ± 0.4 | 5.4 ± 2.9 | 7.9 ± 4.5 | 8.7 ± 2.5 | 4.7 ± 2.5 |
| CuO—PS | 36.0 ± 5.3 | 22.3 ± 4.5 | 25.6 ± 2.3 | 27.7 ± 5.4 | 31.7 ± 4.9 | 21.7 ± 2.9 |
| CuCl₂—PS | 31.7 ± 1.5 | 28.4 ± 0.8 | 30.7 ± 4.1 | 37.4 ± 4.5 | 31.2 ± 4.1 | 42.3 ± 3.1 |
| PS | 55.0 ± 8.7 | 35.4 ± 2.3 | 36.8 ± 0.8 | 38.7 ± 0.7 | 32.4 ± 3.7 | 35.6 ± 3.6 |
| Cu₂O | 61.0 ± 1.7 | 32.4 ± 4.5 | 35.4 ± 2.6 | 37.2 ± 5.4 | 41.6 ± 6.5 | 34.7 ± 7.8 |
| CuCl | 90.3 ± 0.6 | 37.4 ± 3.5 | 38.9 ± 4.5 | 40.9 ± 8.4 | 42.4 ± 2.3 | 52.6 ± 5.4 |
| CuO | 84.7 ± 4.0 | 87.1 ± 2.3 | 79.4 ± 8.9 | 78.1 ± 6.5 | 71.2 ± 8.9 | 72.7 ± 4.0 |
| CuCl₂ | 71.0 ± 1.0 | 81.5 ± 7.4 | 82.6 ± 6.8 | 81.3 ± 5.6 | 81.6 ± 4.5 | 82.0 ± 1.0 |

All the Cu—PS complexes contained 0.07% (w/v) polysaccharide and 30 ppm copper. Values in the table are means ± SD of three different experiments performed in triplicate.

### Example 8

### Influence of Various Copper Compounds on Microbial Growth in the Presence and Absence of Gold

In order to understand the mode of action of the nanospikes on antimicrobial activity the surface effects of the complexes were “neutralized” by exposing the microbial cultures to the Cu-PS complex coated surfaces with an additional layer of gold (Gross-Aviv T.; Vago R. The role of aragonite matrix surface chemistry on the chondrogenic differentiation of mesenchymal stem cells, (2009)). This method causes the spikes to protrude so their activity is more apparent.

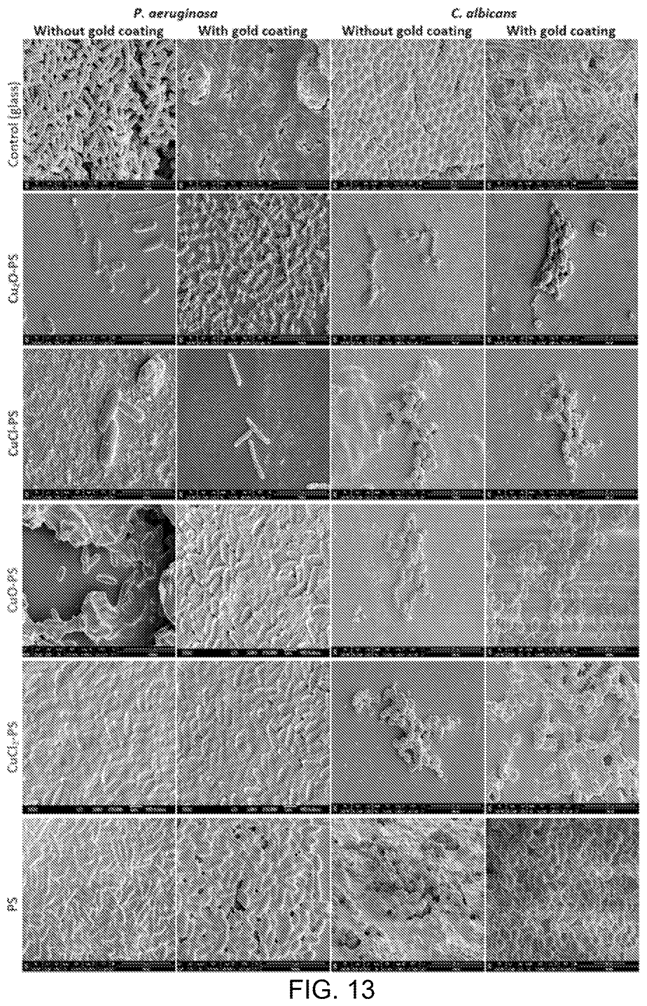

The various surfaces were compared to Cu-PS complexes coated surfaces without additional layer of gold. This comparison is presented in FIG. 13.

FIG. 13 is a series of photomicrographs illustrating the influence of PS only (0.7% w/v), Cu₂O-PS (0.7% w/v PS and 500 ppm copper), CuCl-PS (0.7% w/v PS and 500 ppm copper), CuO-PS (0.7% w/v PS and 500 ppm copper), and CuCl₂-PS (0.7% w/v PS and 500 ppm copper) on growth of P. aeruginosa and C. albicans grown on glass plates with and without an additional layer of gold.

Results presented in FIG. 13 indicate that all Cu-PS complexes were less effective against the fungi model C. albicans and the bacterial model P. aeruginosa when the surfaces were coated with gold. This suggests that the copper valence (complexes' chemistry) have influence on the antimicrobial activity of the complexes and their physical properties alone are Insufficient. In addition, these results indicate that with and without the gold coating to the different surfaces, the Monovalent Cu-complexes are more effective against the two microorganisms than the Divalent Cu-complexes. This suggests that the spikes protrude and their morphology can contribute to inhibition of microbial attachment.

This example illustrates that the Monovalent Cu-complexes as in all their chemical and physical properties have an unexpectedly significant anti-microbial activity which is greater than the sum of monovalent copper alone and polysaccharide alone.

### Example 9

### Role of Nanospikes in Cytotoxicity

Based on AFM and SEM results presented above, it was hypothesized that the appearance of spikes (height, thickness, and density) contributes to prevention of microbial adherence by membrane destruction.

In order to support this hypothesis, the cytotoxic effect of Monovalent Cu-complexes, Divalent Cu-complexes and PS on the E. coli TV1061 reporter strain was assayed. Over the years, bioluminescence of genetically modified bacterial cells has been explored in whole cell biosensor devices in the detection of various toxicants such as pesticides (Hakkila et al., (2004) Detection of bioavailable heavy metals in EILATox-Oregon samples using whole-cell luminescent bacterial sensors in suspension or immobilized onto fibre-optic tips.; Jia et al., (2012) A lower limit of detection for atrazine was obtained using bioluminescent reporter bacteria via a lower incubation temperature.; heavy metals (Ivask et al., (2007) Green T., Polyak B., Mor A., Kahru A., Virta M., Marks R. S. Fibre-optic bacterial biosensors and their application for the analysis of bioavailable Hg and As in soils and sediments from Aznalcollar mining area in Spain; Jouanneau et alk., (2011) Improvement of the Identification of Four Heavy Metals in Environmental Samples by Using Predictive Decision Tree Models Coupled with a Set of Five Bioluminescent Bacteria.; and organic pollutants (Rozen et al., (1999) Specific detection of p-chlorobenzoic acid by Escherichia coli bearing a plasmid-borne fcbA′::lux fusion). Here, the plasmids of bacterial cells are genetically modified to include toxicant-specific promoter genes that regulate the expression of bioluminescence proteins (Girotti et al., (2008) Monitoring of environmental pollutants by bioluminescent bacteria). Escherichia coli strain TV1061 harbors a fusion of luxCDABE reporter gene and the promoter for the heat-shock gene grpE. The grpE gene is sensitive to metabolic changes that can be activated due to the presence of cytotoxic substances like copper.

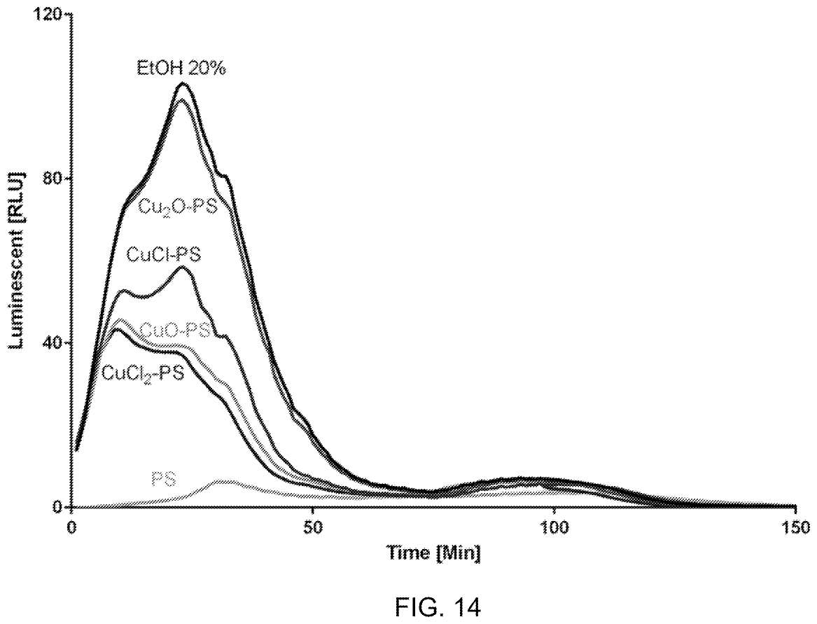

Results are summarized graphically in FIG. 14.

FIG. 14 is a plot of luminescence [RLU] as a function of time in minutes for E. coli TV1061 induced to bioluminescence with: PS only (0.07% w/v; green line), Cu₂O-PS (0.07% w/v PS and 30 ppm copper; red line), CuCl-PS (0.07% w/v PS and 30 ppm copper; brown line), CuO-PS (0.07% w/v PS and 30 ppm copper; light blue line), and CuCl₂-PS (0.07% w/v PS and 30 ppm copper; dark blue line) and 20% ethanol (black line).

Here again the Monovalent Cu-complexes exhibited a more pronounced cytotoxic effect against E. coli TV1061 than the Divalent Cu-complexes and/or PS. The extent of bioluminescence induction of the Monovalent Cu-complex, Cu₂O-PS, was the highest from all complexes and in agreement with that of the membrane-damaging 20% ethanol positive control sample. The other Monovalent Cu-complex, CuCl-PS, and both Divalent Cu-complexes exhibited moderate activity as compared to the control.

These results support a cytotoxic mechanism of the Monovalent Cu-complex, Cu₂O-PS via membrane damage from nanospikes.

### Example 10

### Additional Surface Morphology Study by SEM

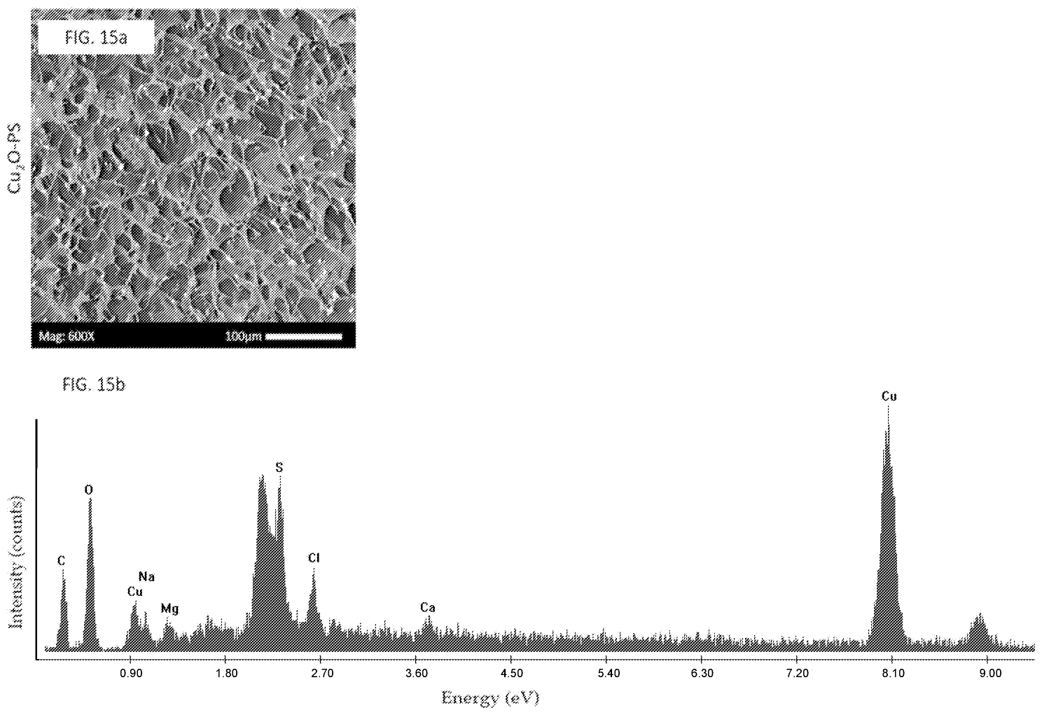

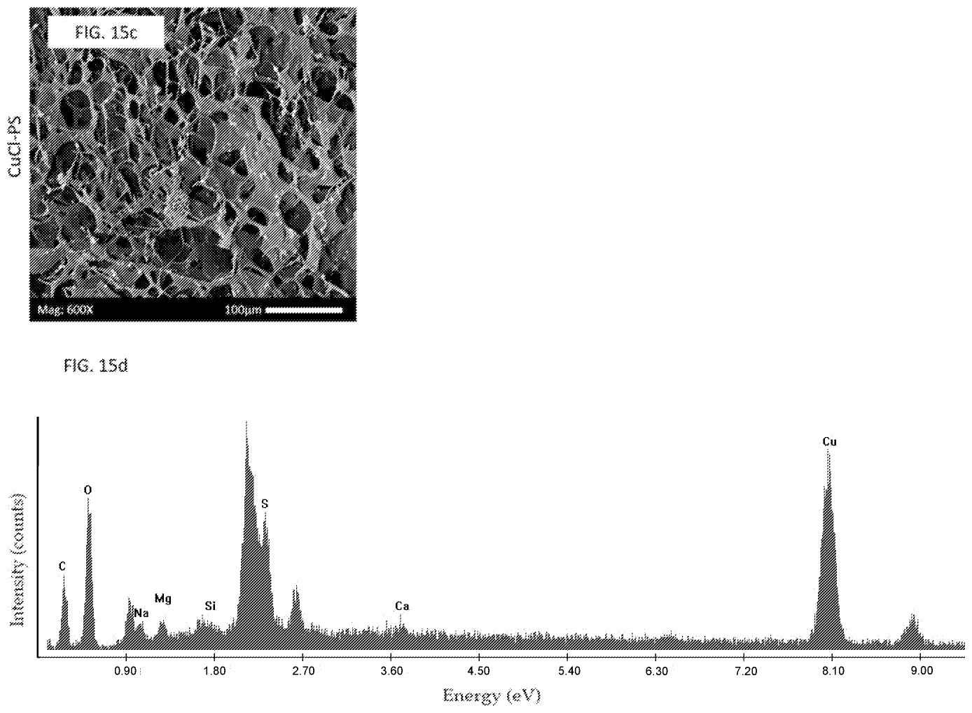

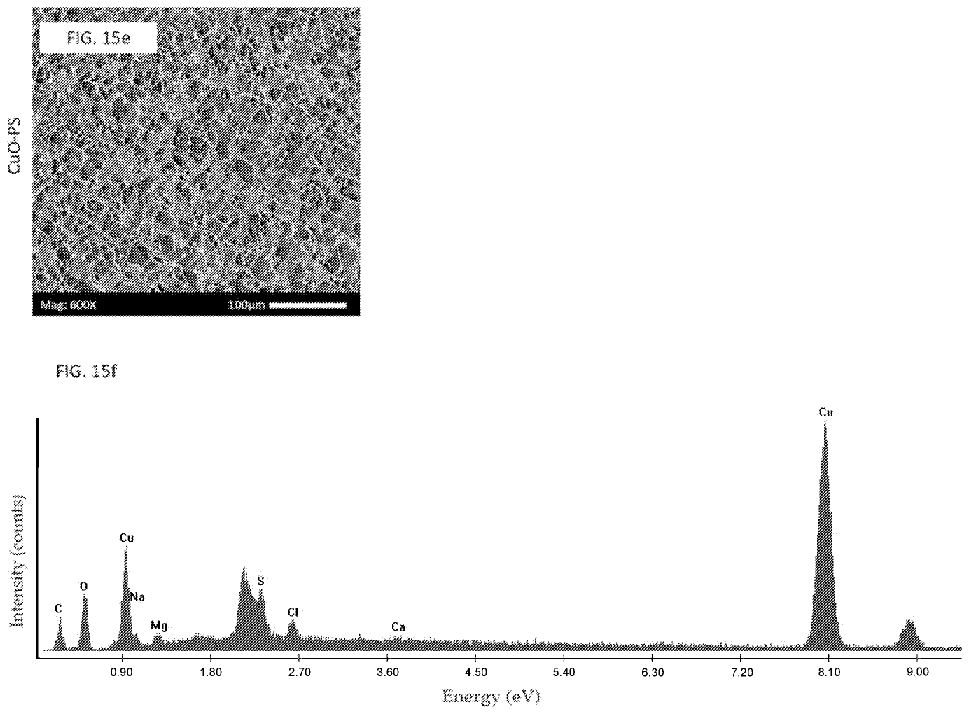

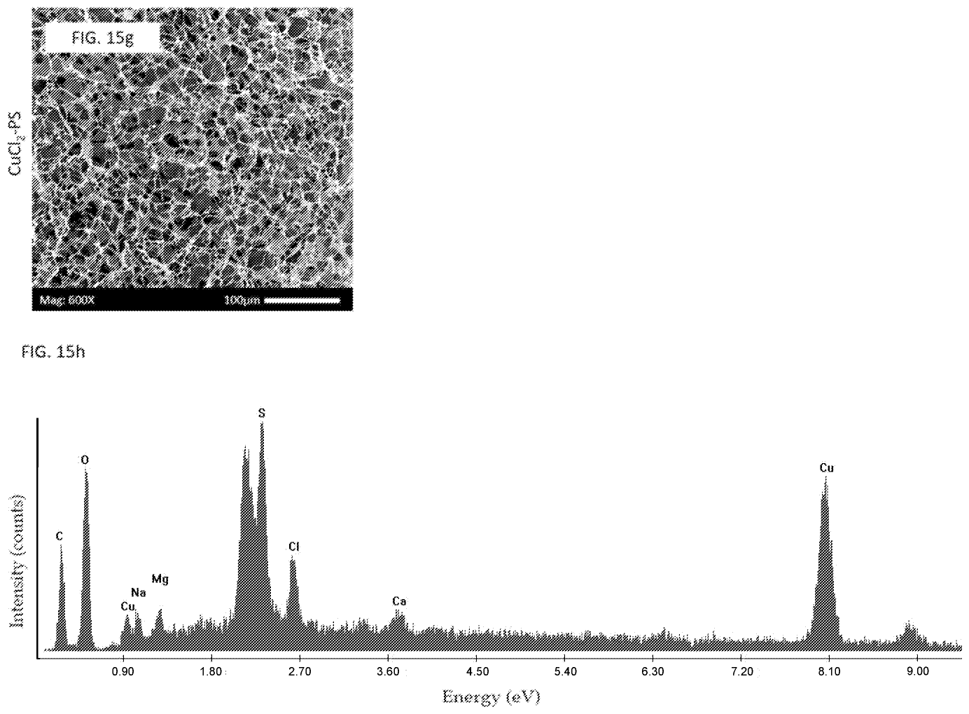

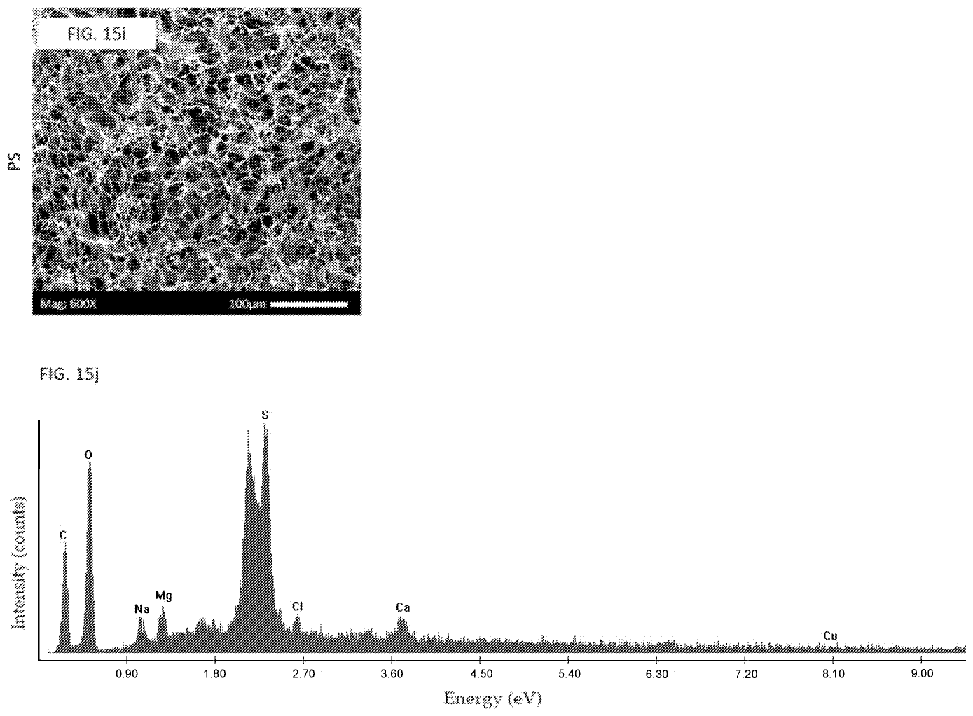

In order to further characterize the surface morphology of the polysaccharide and the Cu-PS complexes an additional SEM study was conducted. Results are presented in FIGS. 15a though 15j.

As can be seen from FIG. 15i the structure of the polysaccharide is porous and fibrous. Differences can be seen in the quantity and in the size of the pores between the polysaccharide and the Monovalent Cu-complexes, with the complexes having fewer entanglements and larger pores. In addition, in corroboration with the AFM results, these complexes showed a distinct morphology with bright, uniformly scattered needle-like structures (spikes). The Divalent Cu-complexes (FIGS. 15e and 15g) were more fibrous than the polysaccharide.

EDS used to analyze the chemical compositions of the Cu-PS complexes and the polysaccharide showed five peaks corresponding to five major elements (FIG. 15b, FIG. 15d, FIG. 15f, FIG. 15h, and FIG. 15j), with the dominant gold peak being due to the gold coating. The main elements identified in the polysaccharide sample and all Cu-PS complexes were carbon and oxygen, as is to be expected for a sugar-containing polymer. In addition, for the PS sample, sulfur and small traces of copper probably originating from the growth medium (which contains microelements including copper). FIG. 15b and FIG. 15d show the elemental composition of Cu₂O-PS complex and CuCl-PS complex detected by EDS. Both cases we notice another dominate peak of copper. FIG. 15f and FIG. 15h show the elemental composition of CuO-PS complex and CuCl₂-PS complex detected by EDS. As expected, the quantities of copper in these complexes are significantly higher than that in the polysaccharide. It is assumed, in II complexes, that the Cu is located on the surface of the complex.

FIG. 15a is an SEM micrograph (600×) of a Cu₂O-PS complex [0.7% (w/v) polysaccharide and 500 ppm copper].

FIG. 15b is an EDS spectrum (Intensity (counts) as a function of Energy (eV)) of the Cu₂O-PS complex of FIG. 15a.

FIG. 15c is an SEM micrograph (600×) of a CuCl-PS complex [0.7% (w/v) polysaccharide and 500 ppm copper].

FIG. 15d is an EDS spectrum (Intensity (counts) as a function of Energy (eV)) of the CuCl-PS complex of FIG. 15c.

FIG. 15e is an SEM micrograph (600×) of a CuO-SP complex [0.7% (w/v) polysaccharide and 500 ppm copper];

FIG. 15f is an EDS spectrum (Intensity (counts) as a function of Energy (eV)) of the CuO-SP complex of FIG. 15e.

FIG. 15g is an SEM micrograph (600×) of a CuCl2-PS complex [0.7% (w/v) polysaccharide and 500 ppm copper]

FIG. 15h is an EDS spectrum (Intensity (counts) as a function of Energy (eV)) of the CuCl₂-PS complex of FIG. 15g.

FIG. 15i is an SEM micrograph (600×) of 0.7% (w/v) polysaccharide (PS).

FIG. 15j is an EDS spectrum (Intensity (counts) as a function of Energy (eV)) of the PS of FIG. 15i.

### Example 11

### Additional Physicochemical Study

In order to expand the physicochemical characterization of example 1 to the other copper/PS complexes, an additional study was conducted.

The viscosity, conductivity, ζ-potential and pH of the Cu-PS complexes (0.7% w/t PS) with 500 ppm copper ions concentrations were determined. The viscosity was measured using Brookfield DV2 viscometer spindle 31, 30rpm. The conductivity and pH determined with a pH/mV/Cond./TDS/Temp. meter 86505 at room temperature. Zeta potential was measured by Zetasizer (Zetasizer Nano ZS, Malvern, Worcestershire, UK) and data were subsequently analyzed using the Smoluchowski model. Data presented are means±standard error of triplicate samples.

Comparison of the physicochemical characteristics of the Monovalent and Divalent Cu-complexes is presented in Table 4. Viscosity and conductivity of the Monovalent Cu-complexes are higher than that of the Divalent Cu-complexes and of PS alone. The copper oxide complexes of the Monovalent and the Divalent Cu-complexes have higher viscosities and conductivities as compared with the copper chloride complexes (ca 10% higher viscosities and 12-57% higher conductivities for copper oxide complexes).

The ζ-potential of the Monovalent Cu-complexes was lower than that of the PS and the Divalent Cu-complexes, indicating better stability. These differences suggest changes in the structure and arrangement of the polysaccharide, perhaps because of the binding of copper ions to the polysaccharide and exposing or screening of the charged residues.

TABLE 4
The Viscosity, Conductivity, Zeta Potential, and pH of Cu-PS Complexes

| | Monovalent Cu-complexes | | Divalent Cu-complexes | | Control |
|---|---|---|---|---|---|
| | Cu₂O-PS | CuCl-PS | CuO-PS | CuCl₂-PS | PS |
| Viscosity [cP] | 2415.3 ± 60.5 | 2230.0 ± 72.1 | 1978.3 ± 24.8 | 1979.0 ± 18.3 | 1733.7 ± 129.8 |
| Conductivity | 3303.0 ± 265.6 | 2930.7 ± 122.2 | 1943.3 ± 50.3 | 1230.0 ± 81.9 | 1043.3 ± 92.9 |
| [μs] | | | | | |
| ζ-potential | −72.5 ± 1.3 | −65.7 ± 4.1 | −43.6 ± 0.5 | −33.9 ± 0.3 | −67.5 ± 2.3 |
| [mV] | | | | | |
| pH | 6.3 ± 0.7 | 6.4 ± 0.9 | 6.1 ± 0.4 | 6.0 ± 0.2 | 5.3 ± 0.4 |

### Example 12

### FT-IR Transmission Spectra

In order to expand the FT-IR characterization of example 1 to the other copper/PS complexes, an additional study was conducted.

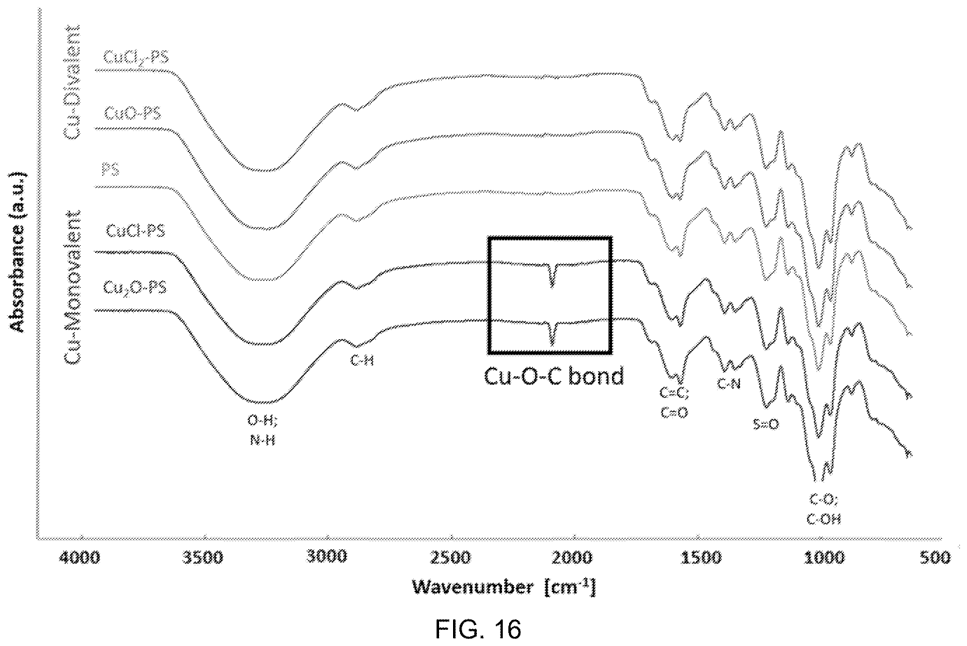

FIG. 16 is an FT-IR transmission spectra (absorbance [a.u] as a function of wavenumber cm⁻¹) of PS (green line) and the Divalent Cu-complexes (CuO-PS and CuCl₂-PS; blue lines) and Monovalent Cu-complexes (Cu₂O-PS and CuCl-PS red lines) in the region of 650-4000 cm−1; all Cu-PS complexes contained 0.7% polysaccharide (w/v) and 500 ppm copper.

To elucidate how the copper binds to the polysaccharide, FT-IR spectroscopy was used (FIG. 16). In the transmission spectrum of the Mono Cu-complexes, a new peak at 1180 cm⁻¹ was observed, suggesting the formation of a new coordinative bond, which was not found in the polysaccharide and the Divalent Cu-complexes. In all spectra, the broad band centered at 3260 cm⁻¹ was assigned to O—H stretching vibrations and the weak signal at 2926 cm⁻¹ to C—H stretching vibrations and the broad band at around 1220-1260 cm⁻¹ was assigned to sulfated ester groups (S═O).

These results confirm the findings of FIG. 1.

1. A composition comprising:
(a) a monovalent copper compound; and
(b) Porphyridium sp. Polysaccharide (PS).

2. The composition according to claim 1, wherein said monovalent copper compound comprises Cu₂O (cuprous oxide).

3. The composition according to claim 1, wherein said monovalent copper compound comprises CuCl (cuprous chloride).

4. The composition according to claim 1, comprising said monovalent copper at a concentration ≥20 PPM.

5. The composition according to claim 1, comprising said monovalent copper at a concentration ≤750 PPM.

6. The composition according to claim 1, comprising said PS at a concentration ≥0.05% (w/v).

7. The composition according to claim 1, comprising said PS at a concentration ≤1.0% (w/v).

8. The composition according to claim 1, provided in a form selected from the group consisting of a spray, a cream, and an ointment.

9. A wound dressing comprising a composition according to claim 1 on an area that contacts a wound.

10. The composition according to claim 1, applied to a medical device.

11. A method comprising:
(a) adding a monovalent copper compound to a solution of Porphyridium sp. Polysaccharide (PS); and
(b) dissolving said monovalent copper compound in said solution to produce an antifungal solution comprising dissolved complex of monovalent copper/PS.

12. The method according to claim 11, comprising sterilizing said antibiotic solution.

13. The method according to claim 11, wherein said monovalent copper compound comprises Cu₂O (cuprous oxide).

14. The method according to claim 11, wherein said monovalent copper compound comprises CuCl (cuprous chloride).

15. The method according to claim 11, wherein said antibiotic solution comprises monovalent copper at a concentration ≥20 PPM.

16. The method according to claim 11, wherein said antibiotic solution comprises monovalent copper at a concentration ≤750 PPM.

17. The method according to claim 11, wherein said antibiotic solution comprises said PS at a concentration ≥0.05% (w/v).

18. The method according to claim 11, wherein said antibiotic solution comprises said PS at a concentration ≤1.0% (w/v).

19. The method according to claim 11, comprising incorporating said antibiotic solution in a member of the group consisting of a spray, a cream, an ointment and a wound dressing at an area that contacts a wound.

20. (canceled)

21. The method according to claim 11, comprising applying said antibiotic solution to a surface.
